(19) United States
(12) Patent Application Publication
(10) Pub. No.: US 2025/0367406 A1
(43) Pub. Date: Dec. 4, 2025

(54) INTRODUCER SHEATH WITH DUAL PORTS

(71) Applicant: BOSTON SCIENTIFIC SCIMED, INC., Maple Grove, MN (US)

(72) Inventors: Neeraj Kumar Sharma, New Delhi (IN); Qian Liu, Plymouth, MN (US); Anurag Sharma, Gurgaon (IN); Boney Augustine, Bhopal (IN)

(73) Assignee: BOSTON SCIENTIFIC SCIMED, INC., Maple Grove, MN (US)

(21) Appl. No.: 19/226,400

(22) Filed: Jun. 3, 2025

Related U.S. Application Data
(60) Provisional application No. 63/655,742, filed on Jun. 4, 2024.

(51) Int. Cl. A61M 25/00; A61M 25/06; A61M 39/06

(52) U.S. Cl. CPC A61M 25/0097; A61M 25/0662; A61M 39/06; A61M 2039/062; A61M 2039/0626; A61M 2039/0633

(57) ABSTRACT

An introducer sheath may include a valve hub and an elongate shaft extending from the valve hub. The valve hub may include a proximal end defining a first port having a first port entry opening and a second port having a second port entry opening. The introduce sheath may include a first seal disposed at the first port and aligned with the first port entry opening and a second seal disposed at the second port and aligned with the second port entry opening. The first port may be parallel with the second port. The valve hub may couple with a stabilizer configured to couple with and axially fix a medical device extending through one or both of the first port and the second port.

### CROSS REFERENCE TO RELATED APPLICATIONS

The present application claims the benefit of U.S. Provisional Patent Application Ser. No. 63/655,742, filed Jun. 4, 2024, the disclosure of which is incorporated herein by reference.

### TECHNICAL FIELD

The present disclosure pertains to sheaths for delivering intravascular medical devices. More specifically, the present disclosure relates to a hub of a sheath with a port for securing and/or sealing medical devices within the hub.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters, guidewires, or other devices, such as a blood pump. A hub may be incorporated at a proximal end of the sheath to provide access to a lumen of an elongate shaft of the sheath. The hub may include one or more seals, e.g., hemostasis seals, to reduce blood leakage as devices are being inserted, positioned, and removed.

### BRIEF SUMMARY

This disclosure provides design, material, manufacturing method, and use alternatives for medical devices, including introducer and/or repositioning sheaths.

A first example is directed to an introducer sheath that may include a valve hub having a proximal end defining a first port having a first port entry opening and a second port having a second port entry opening, an elongate shaft extending from the valve hub, a first seal, the first seal is disposed at the first port and is aligned with the first port entry opening, and a second seal, the second seal is disposed at the second port and is aligned with the second port entry opening, and wherein the first port is parallel to the second port.

Alternatively or additionally to any of the examples above, the first seal may be co-planar with the second seal.

Alternatively or additionally to any of the examples above, the first port entry opening may be co-planar with the second port entry opening.

Alternatively or additionally to any of the examples above, the introducer sheath may further include a third port defined by the valve hub, the third port having a third port entry opening, a third seal, the third seal is disposed at the third port and is aligned with the third port entry opening, and wherein the third port may be parallel with the first port.

Alternatively or additionally to any of the examples above, the first port entry opening may have a first diameter and the second port entry opening may have a second diameter that is smaller than the first diameter.

Alternatively or additionally to any of the examples above, the first port entry opening may be co-axial with the elongate shaft and the second port entry opening may be laterally offset from the first port entry opening.

Alternatively or additionally to any of the examples above, one or both of the first port entry opening and the second port entry opening may include a chamfer narrowing in a distal direction toward the elongate shaft.

Alternatively or additionally to any of the examples above, the valve hub may comprise a body coupled with the elongate shaft and a cap coupled with the body, and wherein the cap may define the first port entry opening and the second port entry opening.

Alternatively or additionally to any of the examples above, the introducer sheath may further include a seal guide positioned between the body and the cap, and wherein the seal guide may be configured to receive the first seal and the second seal to align the first seal with the first port entry opening and align the second seal with the second port entry opening.

Alternatively or additionally to any of the examples above, the introducer sheath may further include one or more walls within the body, and wherein the one or more walls may be configured to direct one or more medical devices received in one or both of the first port and the second port toward a lumen of the elongate shaft.

Alternatively or additionally to any of the examples above, the introducer sheath may further include one or more connectors defined by the valve hub, and wherein the one or more connectors may be configured to engage a stabilizer configured to receive and axially fix a medical device extending through one or both of the first port and the second port.

In another example, an introducer sheath assembly may include a valve hub having a proximal end defining a first port having a first port entry opening and a second port having a second port entry opening, an elongate shaft extending from the valve hub, and a stabilizer configured to couple with the valve hub and engage a medical device extending through the first port entry opening, and wherein the first port may be parallel to the second port.

Alternatively or additionally to any of the examples above, the introducer sheath assembly may further include one or more slots in a proximal facing surface of the valve hub, and wherein the stabilizer may comprise legs and a stabilizer opening, and the legs may be configured to be secured in the one or more slots and the stabilizer opening may be configured to align with the first port entry opening when the legs are secured in the one or more slots.

Alternatively or additionally to any of the examples above, the introducer sheath assembly may further include two or more ridges on opposing sides of the first port entry opening, each of the ridges including a ramped surface facing the first port entry opening, and wherein the stabilizer may comprise a first arm having a first distal end and a second arm may have a second distal end that defines an adjustable opening with the first distal end, and the adjustable opening may be configured to narrow as outer surfaces of the first arm and the second arm engage the two or more ridges.

Alternatively or additionally to any of the examples above, the introducer sheath assembly may further include one or more extensions proximate the first port entry opening, and wherein the stabilizer comprises a stabilizer housing configured to couple with the one or more extensions.

Alternatively or additionally to any of the examples above, the stabilizer may comprise a stabilizer seal having a stabilizer seal opening configured to align with the first port entry opening and a stabilizer seal slot extending from the stabilizer seal opening to an outer circumference of the stabilizer, and the stabilizer may be configured to axially fix the medical device when the stabilizer seal is coupled with the stabilizer housing and the medical device is extending through the stabilizer seal opening.

In another example, a method of positioning one or more medical devices through an introducer sheath having a valve hub may include inserting a first medical device through a first seal of a first port of the valve hub, advancing the first medical device through an elongate shaft of the introducer sheath, inserting a second medical device through a second seal of a second port of the valve hub, the second port may be parallel with the first port, and advancing the second medical device through the elongate shaft of the introducer sheath.

Alternatively or additionally to any of the examples above, the method may further include axially fixing the first medical device relative valve hub by coupling a stabilizer with the first medical device inserted through the first seal and advanced through the introducer sheath.

Alternatively or additionally to any of the examples above, the method may further include coupling a stabilizer with the valve hub.

Alternatively or additionally to any of the examples above, the method may further include adjusting the stabilizer coupled with the valve hub from an unlocked position to a locked position to axially fix the first medical device relative to the valve hub.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic side view of an introducer sheath extending into a blood vessel;

FIG. 2 is a schematic cross-section view of a portion of the introducer sheath of FIG. 1 inserted into a blood vessel, and a medical device inserted into the introducer sheath;

FIG. 3 is a schematic perspective view of an illustrative configuration of an introducer sheath;

FIG. 4 is a schematic exploded side view of various components of the introducer sheath of FIG. 3;

FIG. 5 is a schematic exploded perspective view of various components of the introducer sheath of FIG. 3;

FIG. 6 is a schematic cross-section view of a portion of the introducer sheath of FIG. 3;

FIG. 7 is a schematic cross-section view of the introducer sheath of FIG. 3 with medical devices passing through seals of first and second ports, with the medical devices schematically depicted in a side view;

FIG. 8 is a schematic perspective view of an illustrative configuration of a hub for an introducer sheath;

FIG. 9 is a schematic exploded perspective view of the hub of FIG. 8;

FIG. 10 is a schematic cross-section view of the hub of FIG. 8;

FIG. 11 is a schematic perspective view of an illustrative hub with a stabilizer in a schematic exploded view;

FIG. 12 is a schematic first end view of a cap of the hub depicted in FIG. 11 with the stabilizer coupled with the cap;

FIG. 13 is a schematic second end view of the cap of the hub depicted in FIG. 11 with the stabilizer coupled with the cap;

FIGS. 14A and 14B schematically depict securing a medical device at the hub with the stabilizer depicted in FIG. 11;

FIG. 15 is a schematic perspective view of an illustrative hub with a stabilizer;

FIGS. 16A and 16B schematically depict securing a medical device at the hub with the stabilizer depicted in FIG. 15;

FIG. 17 is a schematic perspective view of an illustrative hub with a stabilizer;

FIG. 18 is a schematic perspective view of the hub with the stabilizer depicted in FIG. 17, with the stabilizer depicted in a schematic exploded view;

FIGS. 19A-19C schematically depict securing a medical device at the hub with the stabilizer depicted in FIG. 17;

FIG. 20 is a schematic perspective view of an illustrative hub with a stabilizer; and

FIG. 21 is a schematic cross-section view of the hub with the stabilizer depicted in FIG. 20.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Hemostasis valve hub assemblies of or provided with an introducer sheath or other suitable sheath may facilitate insertion and positioning of one or more medical devices (e.g., catheters, blood pumps, guidewires, etc.) through the introducer sheath while also helping to prevent blood from leaking during a medical procedure. Some configurations of the present disclosure may include assemblies with components that assist with fixing (e.g., fixing, securing, and/or locking axially, rotationally, etc.) a position of one or more medical devices while maintaining a seal around an elongate shaft of the medical device to reduce blood loss through the hub assembly. Some configurations of the disclosed assemblies may provide components that facilitate convenient repositioning of the one or more medical devices relative to the introducer sheath.

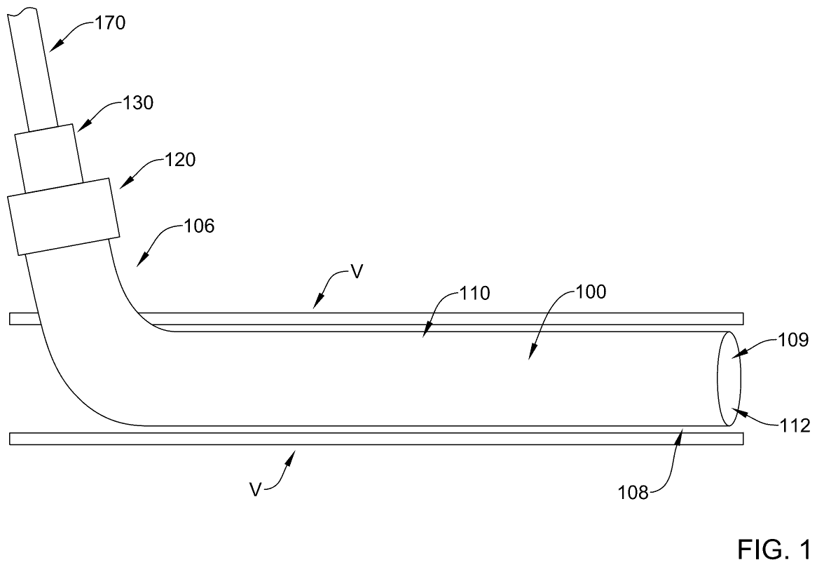

FIG. 1 illustrates a side view of an introducer sheath 100 inserted at least partially into a blood vessel V, shown in cross-section. While the disclosure herein is made with reference largely to the introducer sheath 100, and components thereof, the disclosure may also apply to a repositioning sheath and/or other suitable sheaths.

In some example configurations, the introducer sheath 100 may be used for facilitating the passage of various medical devices, such as catheters, guidewires, blood pumps, and/or other suitable medical devices, through the introducer sheath 100 and into the blood vessel V. In some examples, the introducer sheath 100 may be referred to as a large bore introducer sheath. The introducer sheath 100 may include a proximal end region 106 proximate a proximal end of the introducer sheath 100 and a distal end region 108 proximate a distal end of the introducer sheath 100 that is opposite the proximal end region 106. A body portion 110 of the introducer sheath 100 may extend between the proximal end region 106 and the distal end region 108, and the body portion 110 may entirely or at least partially define a lumen 112 of the introducer sheath 100. The introducer sheath 100 may include a proximal opening (not shown) proximate the proximal end region 106 and a distal opening 109 proximate the distal end region 108, with the lumen 112 extending between the proximal opening and the distal opening 109.

The introducer sheath 100, or components thereof, may be formed by various materials, such as polymeric and/or metallic materials. In some examples, the introducer sheath 100, such as an elongate shaft of the introducer sheath 100, may include a surface coating, such as but not limited to, silicone, PET, or other applicable polymer.

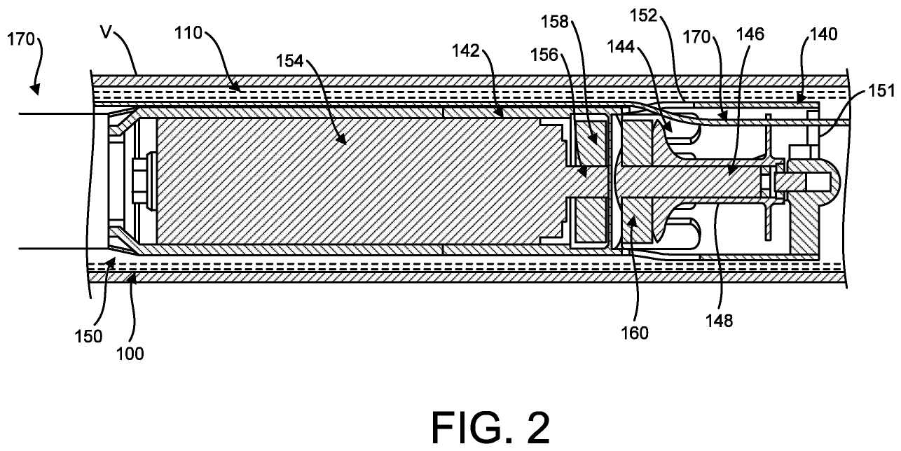

A hemostasis valve hub 120 (hereinafter “hub” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter, blood pump, guidewire, etc. may be inserted through the hub 120 and the lumen 112 of the introducer sheath 100 and into the blood vessel V, and the hub 120 may maintain hemostasis between the medical device 170, the introducer sheath 100, and the external surroundings. In some examples, the medical device 170 may include and/or be coupled to a blood pump 150 (e.g., as shown in FIG. 2).

After insertion of the medical device 170 into and/or through the hub 120, fixation (e.g., securing or locking) of the axial and/or rotational position of the medical device 170 may be desired to ensure that the medical device 170 (and any device coupled thereto) is maintained in a proper position during use. It may also be desired for the medical personnel to reposition the medical device 170 after insertion. As such, the hub 120 may include or may be coupled with one or more tightening ports or stabilizers 130 (e.g., clips, etc.), provided with or attachable thereto, that provides for the fixation of the medical device 170 with respect to the hub 120 and blood vessel V. The hub 120 and tightening port(s) or stabilizers 130 may allow for the repositioning of one or more medical device(s) 170 with respect to the hub 120 and the blood vessel V.

FIG. 2 illustrates a cross-section view of the body portion 110 of the introducer sheath 100 of FIG. 1 upon insertion of a medical device, illustratively a blood pump 150, into the introducer sheath 100 (shown in broken lines in FIG. 2). As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in or at a target location, such as a target cardiac location (e.g., the left ventricle), via the introducer sheath 100. While the introducer sheath 100 is illustrated in FIG. 2 with the use of the blood pump 150, various other medical devices may be used in conjunction with the introducer sheath 100 and the hemostasis valve hub 120.

The blood pump 150 may include one or more components. In some examples, the blood pump 150 may include an impeller assembly housing 140 and a motor housing 142. The impeller assembly housing 140 and the motor housing 142 may be integrally or monolithically constructed. Alternatively, the impeller assembly housing 140 and the motor housing 142 may be separate components. The impeller assembly housing 140 may carry an impeller assembly 144 therein. The impeller assembly 144 may include an impeller shaft 146 and an impeller 148 that rotate relative to the impeller assembly housing 140 to drive blood through the blood pump 150. In some examples, the impeller shaft 146 and the impeller 148 may be integrally formed, whereas, in other examples the impeller shaft 146 and the impeller 148 may be separate components.

Rotation of the impeller 148 may cause blood to flow from a blood inlet 151 formed on the impeller assembly housing 140, through the impeller assembly housing 140, and out of a blood outlet 152 formed on the impeller assembly housing 140. As shown in FIG. 2, the inlet 151 may be formed on an end portion of the impeller assembly housing 140 and the outlet 152 may be formed on a side portion of the impeller assembly housing 140. In other examples, the inlet 151 and/or the outlet 152 may be formed on other portions of the impeller assembly housing 140. In some examples, the impeller assembly housing 140 may be coupled to a distally extending cannula, and the cannula may receive and deliver blood to the inlet 151.

The motor housing 142 may carry a motor 154, and the motor 154 may be configured to rotatably drive the impeller 148 relative to the impeller assembly housing 140. In some examples, the motor 154 may rotate a drive shaft 156 coupled with or to a driving magnet 158. Rotation of the driving magnet 158 may cause rotation of a driven magnet 160 that may be coupled with and/or part of the impeller assembly 144. More specifically, in configurations incorporating the impeller shaft 146, the impeller shaft 146 and the impeller 148 may be configured to rotate with the driven magnet 160. In other configurations, the motor 154 may be coupled to the impeller assembly 144 via other components.

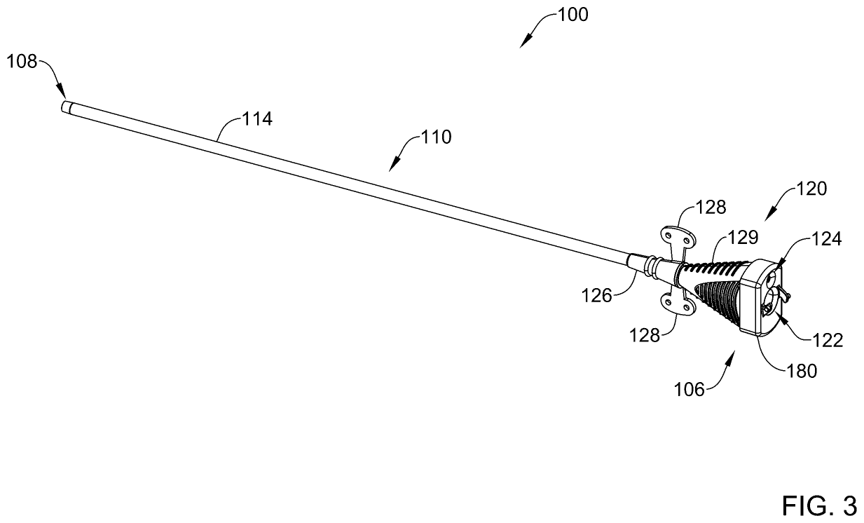

FIG. 3 is a perspective view of an illustrative configuration of the introducer sheath 100. In some examples, the introducer sheath 100 may include the hub 120 and an elongate shaft 114 extending distally from the hub 120 and defining the body portion 110 of the introducer sheath 100.

Although not depicted in FIG. 3, the introducer sheath 100 may include a flush line extending from the hub 120. The flush line may include a tubular member extending from the hub 120 and in fluid communication with a lumen of the hub 120. The tubular member may extend to a stopcock, such as a three-way stopcock, and/or other suitable valve or valve system. The stopcock, when included, may include a first leg coupled to the tubular member, a second leg, a third leg, and a lever that may be rotatable to selectively open/close fluid access between the first, second, and/or third legs. Each of the legs may include a connector, such as a luer connector, as desired.

The hub 120 may include a strain relief 126 configured to provide a flexibility transition along the proximal end region 106. The strain relief 126 may include a body attached to and/or extending from a main body of the hub 120.

The strain relief 126 may include and/or may be coupled with one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The suture pads 128 may be perpendicular to or angled at an acute angle relative to the longitudinal axis of the introducer sheath 100. For example, the suture pads 128 may be angled toward the distal end region 108 of the introducer sheath 100 such that the free ends of the suture pads 128 are positioned distal of the base ends of the suture pads 128. In some examples, the angle between the suture pads 128 and the longitudinal axis of the introducer sheath 100 may be in a range of about 30 degrees to about 90 degrees, about 35 degrees to about 85 degrees, about 40 degrees to about 80 degrees, about 45 degrees to about 75 degrees, about 50 degree to about 70 degrees, or about 55 degrees to about 65 degrees. In some examples, the angle between the suture pads 128 and the longitudinal axis of the introducer sheath 100 may be about 35 degrees, 40 degrees, 45 degrees, 50 degrees, 55 degrees, or 60 degrees.

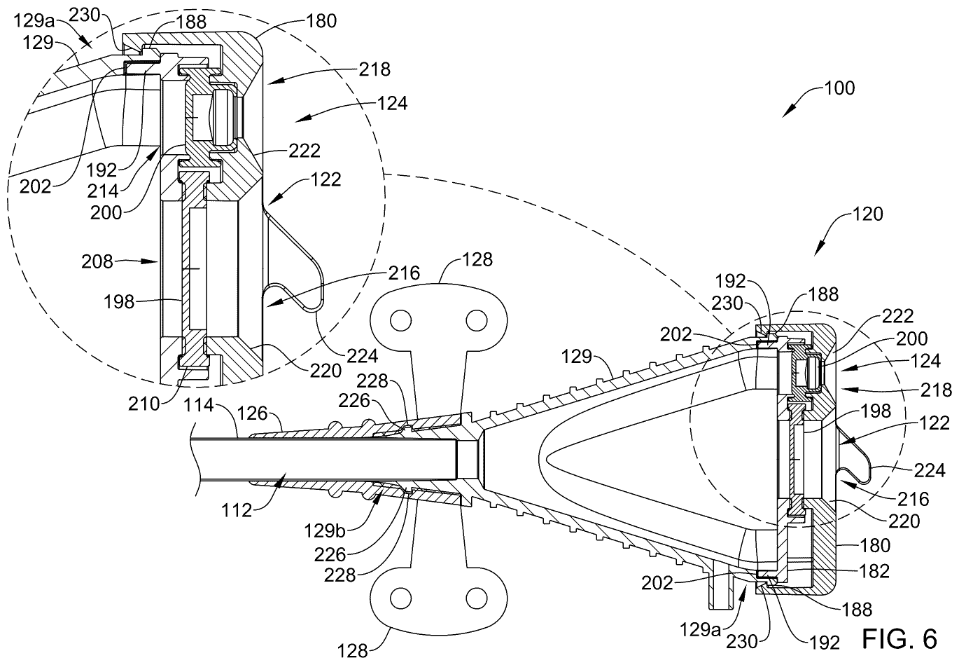

A distal end portion of a hub body 129 of the hub 120 may extend into the interior of the strain relief 126 and/or the suture pads 128 and be coupled thereto (e.g., see FIG. 6). For example, the hub body 129 may include an annular rim configured to engage (e.g., form a snap fit or interference fit) with a mating recess formed in the interior of the strain relief 126 and/or the suture pad(s) 128. In some examples, the suture pad(s) 128 and/or the strain relief 126 may be rotatable relative to the elongate shaft 114 and/or the hub body 129, but other suitable configurations are contemplated.

The hub 120 may include a cap 180 configured to couple with the hub body 129. In some examples, the cap 180 may entirely or at least partially define the first port 122, the second port 124, and/or one or more other suitable ports.

The cap 180 may be configured to couple with the hub body 129 in any suitable manner. For example, the cap 180 may be configured to couple with the hub body 129 via a threaded connection, a snap connection, an interference fit connection, a friction fit connection, a luer connection, and/or one or more other suitable connections. In one example, the cap 180 may couple with the hub body 129 via a snap connection, but other suitable configurations are contemplated.

The first port 122, the second port 124, and/or one or more other suitable ports of or at the hub 120 may have any suitable configuration with respect to one another. In some examples, the first port 122, the second port 124, and/or one or more other suitable ports may be parallel to one another and configured to receive medical devices that are generally parallel to one another as the medical devices enter respective ports. In some examples, the first port 122, the second port 124, and/or one or more other suitable ports may be co-axial with and/or parallel to the elongate shaft 114 (e.g., co-axial with or parallel to at least a proximal end of the elongate shaft 114 proximate the hub 120).

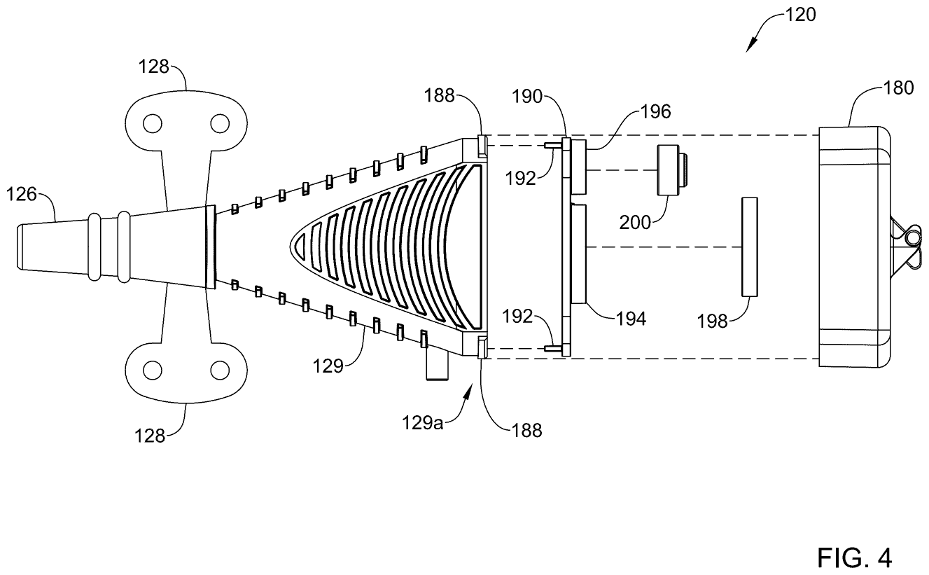

FIG. 4 depicts a schematic side view of the hub 120 depicted in FIG. 3, with various components of the hub 120 in an exploded view. In some examples, the hub 120 may include one or more components monolithically formed together and/or formed as separate components. As depicted in FIG. 4, the hub 120 may include, among other suitable components, the hub body 129, the cap 180, a seal guide 182 (e.g., a seal plate, etc.), and one or more seals (e.g., a first seal 198 and a second seal 200), but other suitable hub configurations and/or components are contemplated.

The hub body 129 may have any suitable configuration. In some examples, the hub body 129 may have an outer surface that tapers distally on at least two sides, one or more indentations, ridges, and/or other suitable components at a proximal end 129a of the hub body 129 that may couple with the cap 180, one or more indentations, ridges, or other suitable components at the proximal end 129a of the hub body 129 that may couple with the seal guide 182, and/or one or more other suitable components. In one example, the hub body 129 may include one or more ridges 188 at or proximate the proximal end 129a of the hub body 129 that are configured to couple with the cap 180 via a snap-fit connection or other suitable connection. To facilitate grasping the hub body 129, an exterior of the hub body 129 may include one or more protrusions, indentations, finger holds, etc. extending along one or more sides of the hub body 129, as desired.

The seal guide 182 may have any suitable configuration. In some examples, the seal guide 182 may have a body 190 (e.g., a plate-like body or other suitable type of body), one or more feet 192 extending distally from the body 190 and configured to engage or otherwise couple with the hub body 129 (e.g., within an indentation, such as a lot, of the hub body 129), a first sub-guide 194 extending proximally from the body 190 and configured to engage or receive a first seal 198 and align the first seal with a first port entry opening discussed herein, a second sub-guide 196 extending proximally from the body 190 and configured to engage or receive a second seal 200 and align the second seal 200 with a second port entry opening discussed herein, and/or one or more other suitable components. In one example, the first sub-guide 194 and the first seal 198 may be part of the first port 122 and the second sub-guide 196 and the second seal 200 may be part of the second port 124. Other suitable configurations of the seal guide 182 are contemplated. In some examples, the seal guide 182 may be omitted and the sub-guide features thereof may be incorporated into the hub body 129 and/or the cap 180.

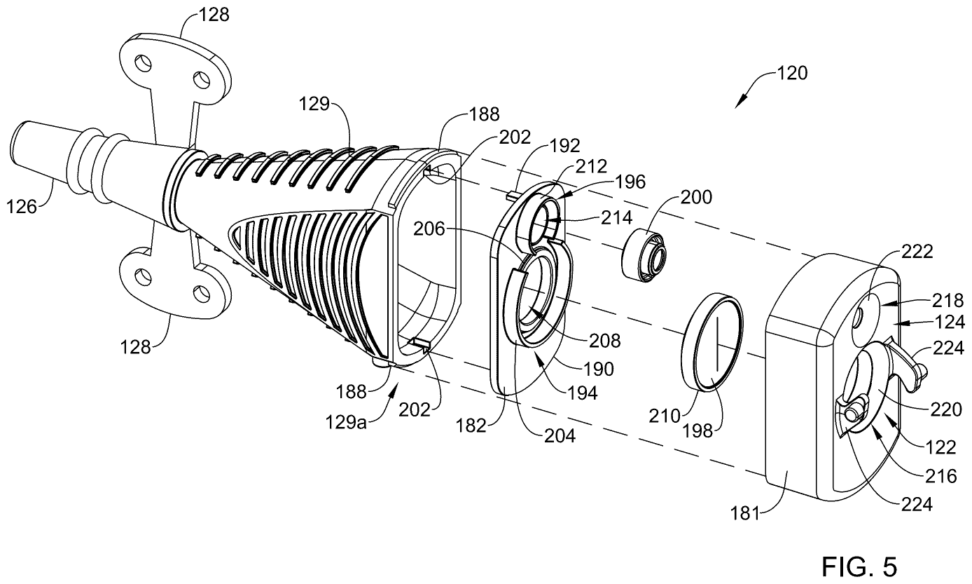

FIG. 5 depicts a schematic perspective view of the hub 120 depicted in FIG. 4. As depicted in FIG. 5, the proximal end 129a of the hub body 129 may include one or more slots 202. In some examples, the one or more slots 202 may be on an interior surface of the hub body 129 and extend distally from a proximal facing end wall of the hub body 129, but other suitable configurations of the slots 202 are contemplated. In one example configuration, the hub body 129 may include two slots 202 on opposing walls, where the slots 202 may be sized and located (e.g., configured to) engage or otherwise receive the distally extending feet 192 of the seal guide 182, but additional or alternative coupling components may be utilized. When the seal guide 182 is coupled with the hub body 129, a distal facing side of the body 190 of the seal guide 182 may abut the proximal facing end of the hub body 129.

The first sub-guide 194 may include any suitable components configured to facilitate receiving the first seal 198 and facilitating a medical device passing through the first seal 198 and/or through the seal guide 182. As depicted in FIG. 5, the first sub-guide 194 may include an annular wall 204 (e.g., one or more walls extending in a full or partial ring) extending proximally from the body 190 of the seal guide 182, an annular ridge 206 (e.g., one or more ridges extending in a full or partial ring) extending proximally from the body 190, an opening 208 extending through the body 190, and/or other suitable components or features. In one example configuration, the annular ridge 206 may be radially inward from the annular wall 204 and the opening 208 may be radially inward from the annular ridge 206. In operation, a space between the annular wall 204 and the annular ridge 206 may be configured to receive an outer ring 210 of the first seal 198 such that a portion of the first seal 198 may rest on a proximal facing surface of the annular ridge 206 and an opening through the first seal 198 (e.g., when a medical device is inserted through the first seal 198) may be aligned with the opening 208 through the first sub-guide 194.

The second sub-guide 196 may include any suitable components configured to facilitate receiving the second seal 200 and facilitating a medical device passing through the second seal 200 and/or through the seal guide 182. As depicted in FIG. 5, the second sub-guide 196 may include an annular wall 212 (e.g., a wall extending in a full or partial ring) extending proximally from the body 190 of the seal guide 182 and an opening 214 extending through the body 190, and/or other suitable components or features. In one example configuration, the opening 214 may be radially inward from the annular wall 212. In operation, a space between the annular wall 212 and opening 214 may be configured to receive the second seal 200 such that a portion of the first seal 198 may rest on a proximal facing surface of the body 190 and an opening through the second seal 200 (e.g., when a medical device is inserted through the second seal 200) may be aligned with the opening 214 through the second sub-guide 196.

The first seal 198 and the second seal 200 may be any suitable types of seal. For example, the first seal 198 and/or the second seal 200 may be an elastomeric seal, a slit valve (e.g., a cross-slit valve), a dome valve, a duckbill valve, and/or any other suitable type of seal or valve configured to seal around an elongate shaft of a medical device when passed therethrough. In one example, the first seal 198 and the second seal 200 may each be a slit valve seal, but other suitable configurations are contemplated.

The first seal 198 and the second seal 200 may be formed from any suitable material. In some examples, the first seal 198 and/or the second seal 200 may be formed from material including, but not limited to, elastomeric material, resilient material, compressible material, flexible material, silicone material, polyurethane material, and or other suitable materials. In one example, the first seal 198 and the second seal 200 may be or may be made from an elastomeric material, but use of other suitable materials are contemplated.

The first seal 198 and the second seal 200 may have any suitable size. In some examples, the first seal 198 and the second seal 200 may have a diameter that is the same as or different than a diameter of the other of the first seal 198 and the second seal 200. In some examples, the first seal 198 may be configured to receive a primary medical device (e.g., a blood pump, etc.) and may have a larger diameter than a diameter of the second seal 200 (e.g., the diameter of the second seal 200 may be smaller than a diameter of the first seal 198) that may be configured to receive a secondary medical device (e.g., a guidewire, catheter, etc.) In one example, the first seal 198 may be sized to receive a medical device having an outer diameter of 12 F or less, 12 F or more, 14 F or more, 15 F or more, 16 F or more, 17 F or more, and/or other suitable outer diameter sizes. In one example, the second seal 200 may be sized to receive a medical device having an outer diameter of 5 F or less, 7 F or less, 9 F or less, 11 F or less, 12 F or less, and/or other suitable sizes of medical devices. The seals 198, 200 may be sized to receive medical devices having other suitable sizes.

The cap 180 may include and/or define one or more port entry openings. For example, the cap 180 may define a first port entry opening 216 associated with the first port 122 and a second port entry opening 218 associated with the second port 124. In some examples, the first port entry opening 216 and the second port entry opening 218 may be configured to align with the first seal 198 and the second seal 200, respectively, when the hub body 129, the seal guide 182, and the cap 180 are coupled together. In some examples, the first port entry opening 216 and the second port entry opening 218 may be co-planar and/or include, define, or lead to a first lumen and a second lumen, respectively, that may be parallel to one another, such that the first port 122 and the second port 124 may be parallel to one another. In one example, the first port entry opening 216 may be co-axial with the elongate shaft 114 (e.g., co-axial with at least a proximal end of the elongate shaft 114) and the second port entry opening 218 may be laterally offset from the first port entry opening 216.

The first port entry opening 216 and the second port entry opening 218 may have any suitable configuration for receiving medical devices. In one example, the first port entry opening 216 may have a first entry chamfer 220 that tapers or narrows in a distal direction toward the elongate shaft 114 and/or the second port entry opening 218 may have a second entry chamfer 222 that tapers or narrows in the distal direction. The first entry chamfer 220 and/or the second entry chamfer 222, when included, may facilitate receiving medical devices in the first port 122 and/or the second port 124, respectively, by funneling the medical device toward the first seal 198 and/or the second seal 200 as the medical device is advanced distally. Although other suitable configurations are contemplated, the first port entry opening 216 and the associated first entry chamfer 220 and/or the second port entry opening 218 and the associated second entry chamfer 222 may extend distally from an outer surface of the cap 180 (e.g., an outer surface facing a proximal direction and/or other suitable outer surface).

The first port entry opening 216 and the second port entry opening 218 may have any suitable size. In some examples, the first port entry opening 216 and the second port entry opening 218 may have a diameter that is the same as or different than a diameter of the other of the first port entry opening 216 and the second port entry opening 218. In some examples and similar to as discussed with respect to the first seal 198 and the second seal 200, the first port entry opening 216 may be configured to receive a primary medical device (e.g., a blood pump, etc.) and may have a larger diameter than a diameter of the second port entry opening 218 (e.g., the diameter of the second port entry opening 218 may be smaller than a diameter of the second port entry opening 218) that may be configured to receive a secondary medical device (e.g., a guidewire, catheter, etc.) In one example, the first port entry opening 216 may be sized to receive a medical device having an outer diameter of 12 F or less, 12 F or more, 14 F or more, 15 F or more, 16 F or more, 17 F or more, and/or other suitable outer diameter sizes. In one example, the second port entry opening 218 may be sized to receive a medical device having an outer diameter of 5 F or less, 7 F or less, 9 F or less, 11 F or less, 12 F or less, and/or other suitable sizes of medical devices.

The cap 180 may include one or more connectors 224. The connectors 224, when included, may be any suitable type of connector configured to couple a component to or with respect to the hub 120 and/or the cap 180. For example, the connector 224 may be or include one or more projections, one or more bayonet connector, one or more luer connectors, one or more slots, one or more ramped surfaces, and/or other suitable types of connectors. In one example, the connector 224 may be a bayonet connector (e.g., as depicted in FIG. 5) configured to mate, via a bayonet connection, with one or more components (e.g., a tightening port or stabilizer 130 (e.g., a clip, etc.)).

The cap 180 may have any suitable exterior shape. In some examples, the cap 180 may have one or more planar sides 181, as depicted for example in FIG. 5. In one example, the cap 180 may have at least two planar sides 181, where two planar sides 181 may oppose one another. Utilizing one or more planar sides 181 on the cap 180 may facilitate resting the hub 120 on the patient or a flat surface during use.

FIG. 6 depicts a schematic cross-section view of a portion of the introducer sheath 100 depicted in FIG. 3. As depicted in FIG. 6, the elongate shaft 114 (e.g., a proximal end of the elongate shaft 114) may extend into the strain relief 116 and a distal end 129b of the hub body 129, where the suture pads 128 may extend from strain relief 126, but other suitable configurations are contemplated. In some examples, the strain relief 126 may be releasably coupled with the distal end 129b of the hub body 129 via a snap or interference fit connection or other suitable connection that may allow the strain relief 126 and/or the suture pads 128 to rotate relative to the hub body 129. For example, one or both of the strain relief 126 and the distal end 129b of the hub body 129 may include one or more projections and/or one or more recesses or indentations configured to facilitate coupling the strain relief 126 with the hub body 129 and rotating the strain relief 126 and/or the suture pads 128 relative to the hub body 129. In one example, an exterior surface of the hub body 129 may include or define a protrusion 226 (e.g., an annular ridge extending circumferentially around the hub body 129 and/or other suitable protrusion) and an interior surface of the strain relief 126 may include a recess 228 configured to receive the protrusion 226 to couple the strain relief 126 with the hub body 129 and facilitate rotation of the strain relief 126 and/or the suture pads 128 relative to the hub body 129, but other suitable configurations are contemplated.

In the configuration of the hub 120 depicted in FIG. 6, the hub body 129 may, generally, be hollow such that the interior surfaces of the exterior walls of the hub body 129 may guide medical devices received via the first port 122 and/or the second port 124 toward the lumen 112 of the elongate shaft 114. Alternatively or additionally, the hub body 129 may include one or more walls and/or other components extending from the interior surface of the exterior walls of the hub body 129 to direct medical devices received in the hub body 129 toward the lumen 112 of the elongate shaft 114.

As discussed, the seal guide 182 may couple with the proximal end 129a of the hub body 129. In some examples, the feet 192 of the seal guide 182 may insert into or be received in the slots 202 in the proximal end 129a of the hub body 129 to couple the seal guide 182 with the hub body 129. Other suitable configurations are contemplated.

The cap 180 may be configured to couple with the proximal end 129a of the hub body 129. Although other suitable configurations are contemplated, the cap 180 may include one or more feet 230 configured to engage the ridge(s) 188 on the hub body 129, as depicted for example in FIG. 6. When the feet 230 of the cap 180 engage the ridge 188 of the hub body 129, the cap 180 may be coupled with the hub body via a snap or interference connection with the cap 180 engaging the first seal 198 and the second seal 200 (e.g., engaging proximal facing sides of the first seal 198 and the second seal 200).

When the seal guide 182 is coupled with the hub body 129 and the cap 180 is coupled with the hub body 129, the first seal 198 and the second seal 200 may be located between (e.g., sandwiched between) the seal guide 182 and the cap 180. In some examples, the seal guide 182 and the cap 180 may engage the first seal 198 and the second seal 200 such that the connections between the seal guide 182, the cap 180, the first seal 198, and the second seal 200 are fluid tight. When the seals 198, 200, the seal guide 182, and the cap 180 are coupled together, the seal guide 182, the opening 208 through the seal guide 182, the first seal 198, the first port entry opening 216, and/or other suitable components may form the first port 122 and the seal guide 182, the opening 214 through the seal guide 182, the second seal 200, the second port entry opening 218, and/or other suitable components may form the second port 124. Other suitable configurations of the first port 122 and/or the second port 124 are contemplated.

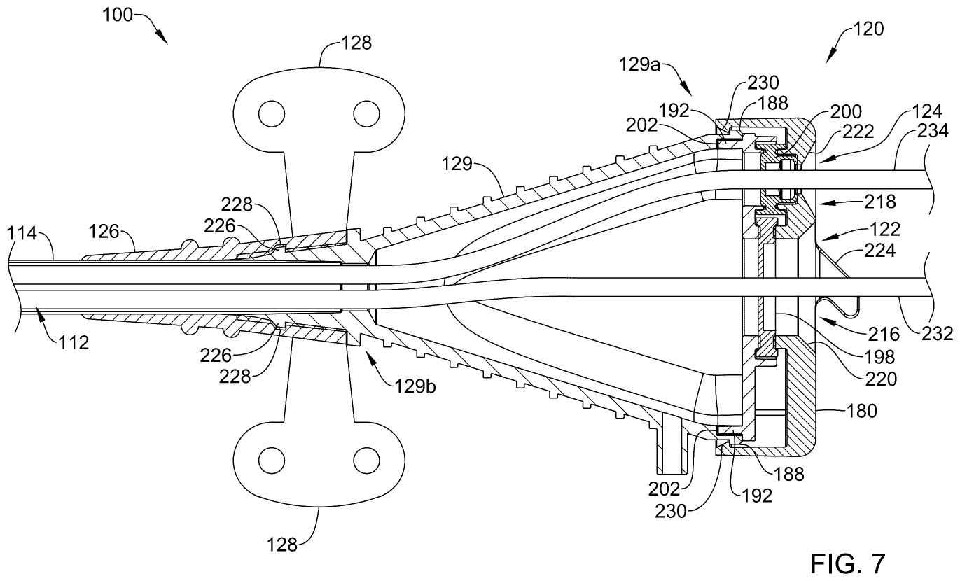

FIG. 7 depicts the schematic cross-section view of the hub 120 depicted in FIG. 6, with medical devices inserted into and through the first port 122 and the second port 124 and advanced through the elongate shaft 114. In some examples, a first medical device 232 (e.g., a blood pump or other suitable medical device) may be inserted through the first seal 198 situated in the first port 122, inserted through the hub 120 and into the elongate shaft 114, advanced through the elongate shaft 114, and advanced to a target location within a patient. Once the first medical device 232 has been inserted through the first port 122, the first seal 198 may fluidly seal around the first medical device 232 to prevent leakage of fluid from the first port 122. A second medical device 234 may be inserted through the second seal 200 situated in the second port 124, inserted through the hub and into the elongate shaft 114, advanced through the elongate shaft 114, and advanced to a target location within the patient, where the target location at which the second medical device 234 is located may be the same or different than the target location at which the first medical device 232 may be located. Once the second medical device 234 has been inserted through the second port 124, the second seal 200 may fluidly seal around the second medical device 234 to prevent leakage of fluid from the second port 124.

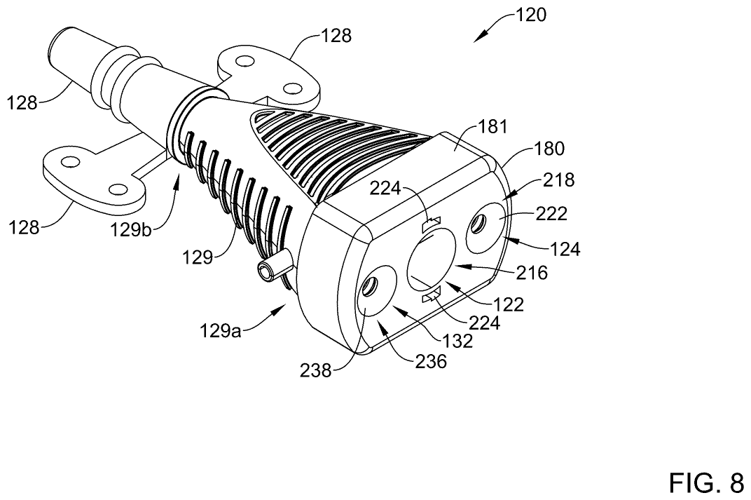

FIG. 8 depicts a schematic perspective view of an illustrative configuration of the hub 120, where the hub 120 comprises the first port 122, the second port 124, and a third port 132. As depicted in FIG. 8, the first port 122 may be centered on the hub 120, with the second port 124 and the third port 132 located to the sides of the first port 122. In some examples, the first port 122 may be configured to receive a primary medical device (e.g., a blood pump and/or other suitable medical device) and the second port 124 and/or the third port 132 may be configured to receive secondary medical devices (e.g., a guidewire, catheter, etc.) Other suitable positioning of the first port 122, the second port 124, and/or the third port 132 are contemplated.

The first port 122, the second port 124, and the third port 132 may include port entry openings, which may be defined by the cap 180. For example, the first port 122 may include the first port entry opening 216, the second port 124 may include the second port entry opening 218, and the third port 132 may include a third port entry opening 236. In some examples, the first port entry opening 216, the second port entry opening 218, and/or the third port entry opening 236 may be co-planar with one another and/or may include, define, or lead to a first lumen, a second lumen, and a third lumen, respectively, that may be parallel to one another, such that the first port 122, the second port 124, and/or the third port 132 may be parallel to one another.

The first port entry opening 216, the second port entry opening 218, and the third port entry opening 236 may have any suitable configuration for receiving medical devices. In one example, the second port entry opening 218 may have a second entry chamfer 222 and/or the third port entry opening 236 may have a third entry chamfer 238, while the first port entry opening 216 may not have a chamfer (e.g., the first entry chamfer 220 is omitted). Although other suitable configurations are contemplated the first port entry opening 216, the second port entry opening 218 and the associated second entry chamfer 222, and/or the third port entry opening 236 and the associated third entry chamfer 238 may extend distally from an outer surface of the cap 180 (e.g., an outer surface facing a proximal direction and/or other suitable outer surface), as depicted for example in FIG. 8. The second entry chamfer 222, and/or the third entry chamfer, when included, may facilitate receiving medical devices in the first port 122, the second port 124, and/or the third port 132, respectively, by funneling the medical device toward seals of the second port 124 and/or the third port 132 as the medical device(s) are advanced distally.

As discussed, the cap 180 may include one or more connectors 224. In one example, the connector(s) 224 may be or may include one or more slots in the cap 180, for example as depicted in FIG. 8, that are configured to mate with or receive one or more components (e.g., one or more components of a tightening port or stabilizer 130 (e.g., a clip, etc.)). Other suitable configurations of the connectors 224 are contemplated.

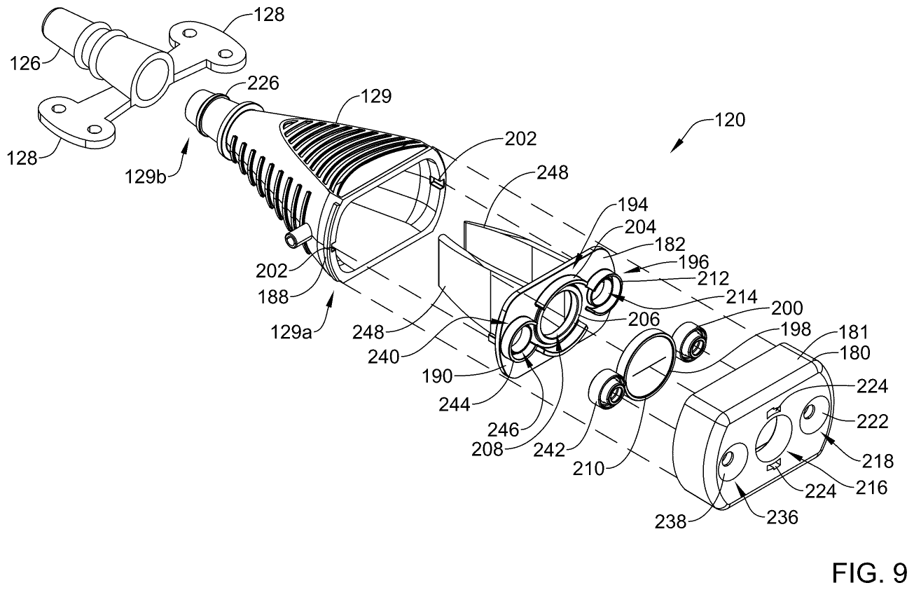

FIG. 9 depicts a schematic perspective exploded view of the configuration of the hub 120 depicted in FIG. 8. In addition to including the first sub-guide 194 configured to receive the first seal 198 and the second sub-guide 196 configured to receive the second seal 200, the seal guide 182 may include a third sub-guide 240 configured to receive a third seal 242 of or for positioning in the third port 132 (e.g., as labeled in FIG. 8). The third sub-guide 240 may include any suitable components configured to facilitate receiving the third seal 242 and facilitating a medical device passing through the third seal 242 and/or through the seal guide 182. As depicted in FIG. 9, the third sub-guide 240 may include an annular wall 244 (e.g., one or more walls extending in a full or partial ring) extending proximally from the body 190 of the seal guide 182 and an opening 246 extending through the body 190, and/or other suitable components or features. In one example configuration, the opening 246 may be radially inward from the annular wall 244. In operation, a space between the annular wall 244 and the opening 246 may be configured to receive the third seal 242 such that a portion of the third seal 242 may rest on a proximal facing surface of the body 190 and an opening through the third seal 242 (e.g., when a medical device is inserted through the third seal 242) may be aligned with the opening 246 through the third sub-guide 240. In some examples, the third seal 242 positioned within the third sub-guide 240 may be aligned with the third port entry opening 236 when the hub body 129, the seal guide 182, and the cap 180 are coupled together with the third seal 242 between the seal guide 182 and the cap 180.

The third seal 242 may be any suitable type of seal. For example, the third seal 242 may be an elastomeric seal, a slit valve (e.g., a cross-slit valve), a dome valve, a duckbill valve, and/or any other suitable type of seal or valve configured to seal around an elongate shaft of a medical device when passed therethrough. In one example, the third seal 242 may be a slit valve seal, but other suitable configurations are contemplated.

The third seal 242 may be formed from any suitable material. In some examples, the third seal 242 may be formed from material including, but not limited to, elastomeric material, resilient material, compressible material, flexible material, silicone material, polyurethane material, and or other suitable materials. In one example, the third seal 242 may be or may be made from an elastomeric material, but use of other suitable materials are contemplated.

The third seal 242 and/or the third port entry opening 236 may have any suitable size. In some examples, third seal 242 and/or the third port entry opening 236 may have a diameter that is the same as or different than a diameter of the first seal 198 or first port entry opening 216 and/or the second seal 200 or the second port entry opening 218. In some examples, the third seal 242 may be configured to receive a secondary medical device (e.g., a guidewire, catheter, etc.) In one example, the third seal 242 may be sized to receive a medical device having an outer diameter of 5 F or less, 7 F or less, 9 F or less, 11 F or less, 12 F or less, and/or other suitable sizes of medical devices.

The seal guide 182 may include one or more walls 248 (e.g., guides) and/or other suitable dividers or path definers extending distally from the body 190 of the seal guide 182. The walls 248 may take on any suitable configuration designed to direct medical devices received in the first port 122, the second port 124, and/or the third port 132 (e.g., as labeled in FIG. 8) to or toward the lumen 112 of the elongate shaft 114 (not shown in FIG. 9). In some examples, the walls 248 may have a length and width suitable for preventing interaction between medical devices received in the hub 120 prior to the medical devices advancing distally past a distal end of the walls 248, which may mitigate entangling of the received medical devices. In one example, the seal guide 182 may include two walls 248 extending distally from the body 190 of the seal guide 182, where distal end portions of the walls 248 may be bent inward toward one another to facilitate guiding medical devices received in the hub 120 toward the elongate shaft 114. Other suitable configurations of the walls 248 and/or other suitable dividers or path definers are contemplated.

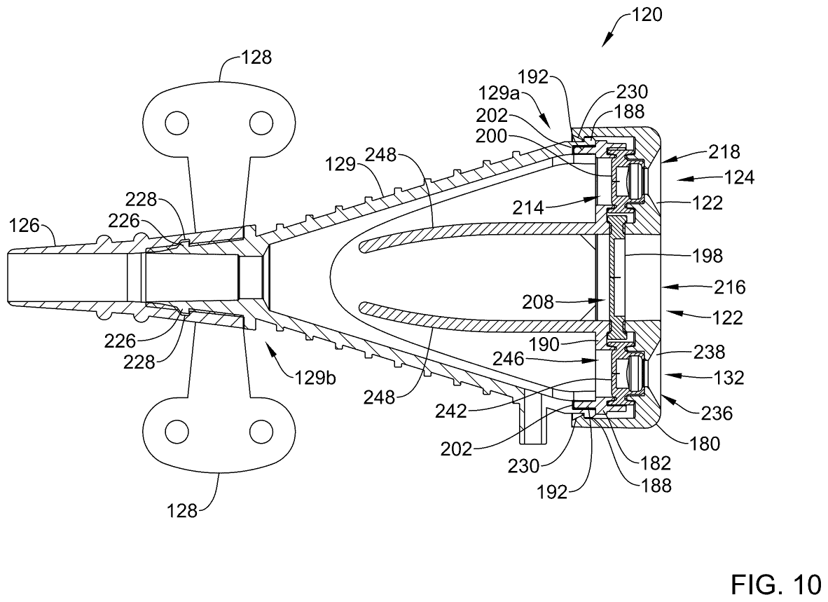

FIG. 10 depicts a schematic cross-section view of the hub 120 depicted in FIG. 8. In the configuration of the hub 120 depicted in FIG. 10, the hub body 129 may, generally, be hollow such that the interior surfaces of the exterior walls of the hub body 129 may guide medical devices received via the first port 122, the second port 124, and/or the third port 132 toward the lumen 112 of the elongate shaft 114 (not shown in FIG. 10). In some examples, the walls 248 extending distally from the body 190 of the seal guide 182 may work with the interior surfaces of the exterior walls of the hub body 129 to guide medical devices received in the first port 122, the second port 124, and/or the third port 132 toward a distal end of the hub 120 and to the elongate shaft 114. Alternatively or additionally, the hub body 129 may include one or more walls and/or other components or features extending from the interior surfaces of the exterior walls or other walls of the hub body 129 to direct medical devices received in the hub body 129 toward the elongate shaft 114.

When the seal guide 182 is coupled with the hub body 129 and the cap 180 is coupled with the hub body 129, the first seal 198, the second seal 200, and the third seal 242 may be located between (e.g., sandwiched between) the seal guide 182 and the cap 180. In some examples, the seal guide 182 and the cap 180 may engage the first seal 198, the second seal 200, and the third seal 242 such that the connections between the seal guide 182, the cap 180, the first seal 198, the second seal 200, and the third seal 242 are fluid tight. When the seals 198, 200, 242, the seal guide 182, and the cap 180 are coupled together, the seal guide 182, the opening 246 through the seal guide 182, the third seal 242, the third port entry opening 236, and/or other suitable components may form the third port 132. Such a configuration of the third port 132 may be parallel or substantially parallel with the first port 122 and/or the second port 124. In some examples, the first seal 198, the second seal 200, and/or the third seal 242 may be co-planar with one or more of the other seals 198, 200, 242 (e.g., as depicted in FIG. 10). Other suitable configurations of the first port 122, the second port 124, and/or the third port 132 are contemplated.

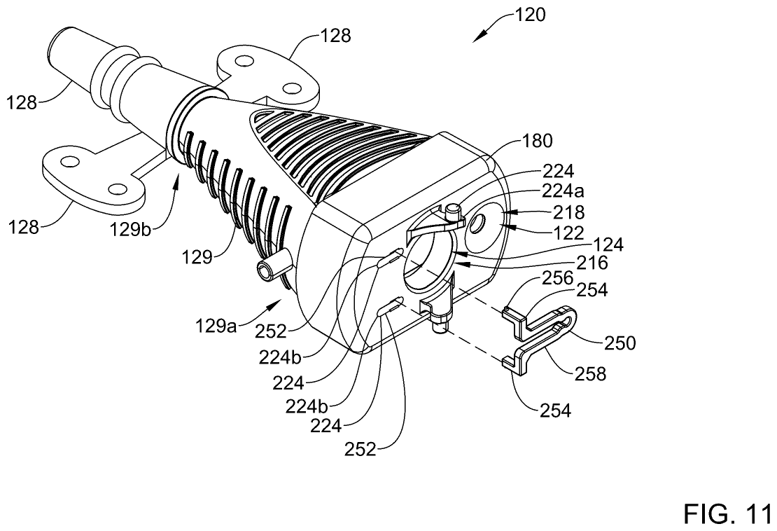

FIG. 11 is a schematic perspective view of an illustrative configuration of the hub 120, with an illustrative configuration of a stabilizer 250 (e.g., which may be a tightening port or stabilizer 130 (e.g., a clip, etc.)) exploded out from the hub 120. As depicted in FIG. 11, the hub 120 may include the first port 122 and the second port 124, along with two sets of connectors 224 at or in a proximal facing surface of the hub 120. In some examples, the cap 180 of the hub 120 may include a first set 224a of connectors 224 with the bayonet connector configuration and a second set 224b of connectors 224 with a slot connector configuration. In one example, when the first set 224a of connectors 224 and the second set 224b of connectors 224 are utilized as depicted in FIG. 11, the first set 224a of connectors 224 may be configured to couple with a dilator hub (not shown) and the second set 224b of connectors 224 may be configured to couple with the stabilizer 250 (e.g., a clip, etc.)

The second set 224b of the connectors 224 may have any suitable slot connector configuration that facilitates coupling the stabilizer 250 with the hub 120. In some examples, the connectors 224 may include one or more slots 252 in the cap 180 that are configure to receive the stabilizer 250 and couple the stabilizer 250 with the cap 180. In some examples, the slots 252 may extend through the cap 180 from a proximal facing surface of the cap 180 and the hub 120 to a distal facing surface. In one example, the second set 224b of the connectors 224 may include two slots 252 that are spaced from one another and parallel to one another, but other suitable configurations are contemplated.

The stabilizer 250 may have any suitable configuration configured to couple with the hub 120 (e.g., the cap 180 and/or other suitable component of the hub 120) and engage a medical device extending through one of the ports of the hub 120 (e.g., the first port 122, the second port 124, the third port 132, when included, etc.). In some examples, the stabilizer 250 may be configured to couple with the second set 224b of connectors 224 depicted in FIG. 11 and align with the first port 122 to engage a medical device extending therethrough when coupled with the second set 224b of connectors. Although the stabilizer 250 may have other suitable configurations, the stabilizer 250 may have a clip configuration.

The stabilizer 250 may include one or more legs 254, one or more feet 256 extending from the one or more legs 254, and one or more arms 258 extending from the legs 254, as depicted for example in FIG. 11. The one or more legs 254 may extend axially and may be configured to extend into and through the slots 252. The one or more arms 258 may extend perpendicular and/or at one or more other suitable angle relative to the one or more legs 254 and in a lateral direction toward the first port 122 when the legs 254 and/or the feet 256 are coupled with the hub 120.

In one example configuration, the stabilizer 250 may include two legs 254, with each leg 254 configured relative to the other so as to extend within one of the slots 252. Each leg 254 may include one or more of the feet 256, where the feet 256 in combination with the legs 254 may be configured to extend into the slots 252 and secure the stabilizer 250 at a desired location relative to the hub 120 (e.g., relative to the first port 122). In some examples, the legs 254 and/or the feet 256 may be flexible and/or resilient to facilitate being inserted into the slots 252 and coupling with the hub 120.

The one or more arms 258 may have any suitable configuration. In some examples, the one or more arms 258 may include an arm 258 with a first end coupled with one leg 254 and a second end coupled with another leg 254, as depicted for example in FIG. 11. In one example, the arm 258 may be U-shaped, but other suitable configurations are contemplated. In some examples, a portion of the arms 258 may be flexible and/or resilient to facilitate receiving a medical device therein or therebetween and/or to facilitate inserting the legs 254 and/or feet 256 into the slots 252. In another example, the one or more arms 258 may include a first arm 258 having a first end coupled with one of the legs 254 and a second end aligned with and proximate the first port 122 and a second arm 258 having a first end coupled with another of the legs 254 and a second end aligned with and proximate the first port 122, where the second ends of the two arms 258 may be configured relative to each other so as to be configured to engage and secure a medical device extending through the first port 122 at a desired location.

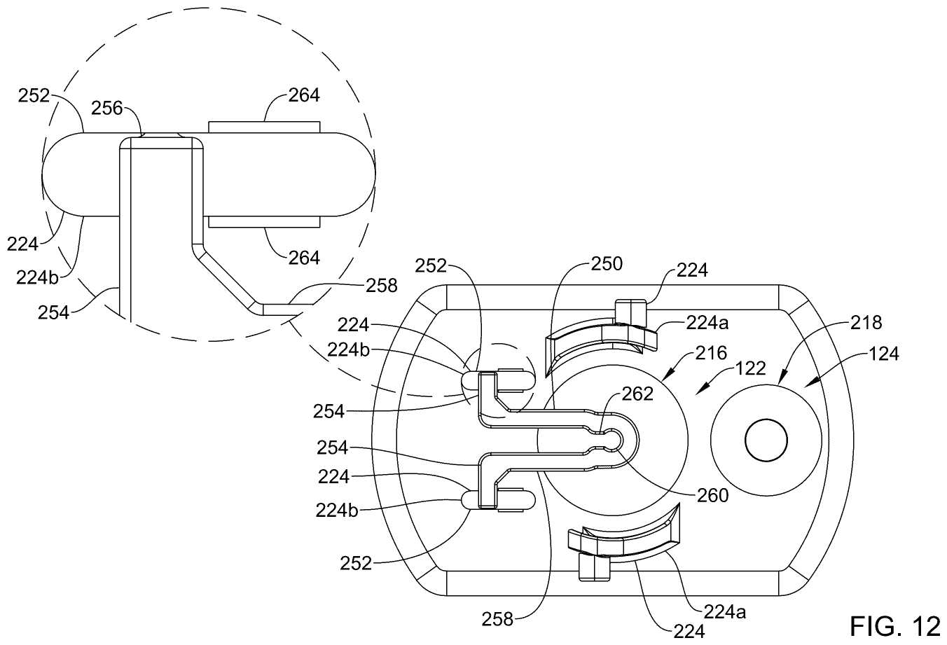

FIG. 12 depicts a schematic first end view (e.g., looking distally) of the stabilizer 250 depicted in FIG. 11 engaged with the cap 180. As depicted in FIG. 12, the stabilizer 250 may be in a locked position within the slots 252 such that a securing portion 260 of the stabilizer 250 (e.g., a stabilizer opening) is aligned with (e.g., co-axial with) a central axis extending through the first port entry opening 216 of the first port 122, but other suitable positioning of the securing portion 260 when the stabilizer 250 is in a locked position is contemplated.

The arm(s) 258 may include one or more locking portions 262 configured to work with the securing portion 260 to secure a medical device at an axial location relative to the stabilizer 250 and/or the hub 120. In one example, the one or more locking portions 262 may include one or more protrusions extending on portions of the arm(s) 258 facing one another and/or offset from one another. When the stabilizer 250 is receiving a medical device in the securing portion 260 (e.g., an opening defined by the arms 258 and/or the locking portions 262), the medical device may need to cross the locking portion(s) 262 to be positioned in (e.g., secured in) the securing portion 260. In some examples, the locking portion(s) 262 may be protrusions of the arm(s) 258 and one or both of the medical device and the arm(s) 258 may need to deflect or bend to cross the locking portion 262 and be positioned in the securing portion 260 of the stabilizer 250. Once in the securing portion 260, the one or both of the medical device and the arm(s) 258 may need to deflect or bend to release or disengage the stabilizer 250 from the medical device.

The securing portion 260 of the arm may have any suitable size or configuration. In some examples, the arm(s) 258 at the securing portion 260 may define an opening configured to receive the medical device and apply sufficient force to the outer surface of the medical device so as to prevent axial and/or rotational movement of the medical device, but not so much force that the medical device is damaged by the force. In one example, the arm(s) 258 may define the opening at the securing portion 260 to have a diameter that is configured to be less than an outer diameter of the medical device received therein, but other suitable configurations are contemplated.

The slots 252 may include one or more features configured to facilitate receiving the legs 254 and/or the feet 256 of the stabilizer 250. In some examples, the slots 252 may include one or more sloped regions 264 configured to facilitate receiving the legs 254 and the feet 256 of the stabilizer 250. Although two sloped regions 264 are depicted at each slot 252 in FIG. 12, it is contemplated that only a single sloped region 264 may be utilized, where the single sloped region 264 may be position along the slot 252 at a location at which the feet 256 of the stabilizer would engage the portion of the cap 180 defining the slot 252 when the stabilizer is in an unlocked position relative to the medical device extending through the first port 122 or other suitable port. In some examples, the sloped regions 264 may be beveled or chamfered surfaces of the cap 180.

In operation, as the legs 254 and the feet 256 are inserted into the slots 252, the sloped regions 264 may engage the legs 254 and/or the feet 256 and cause the stabilizer 250 to bend and move the legs 254 closer to one another as the stabilizer is inserted into the slots 252. Once the feet 256 of the stabilizer are inserted fully through slots 252, the stabilizer 250 may partially or fully relax, depending on a spacing of the slots from one another relative to a spacing of the legs 254 from one another and/or one or more other suitable factors.

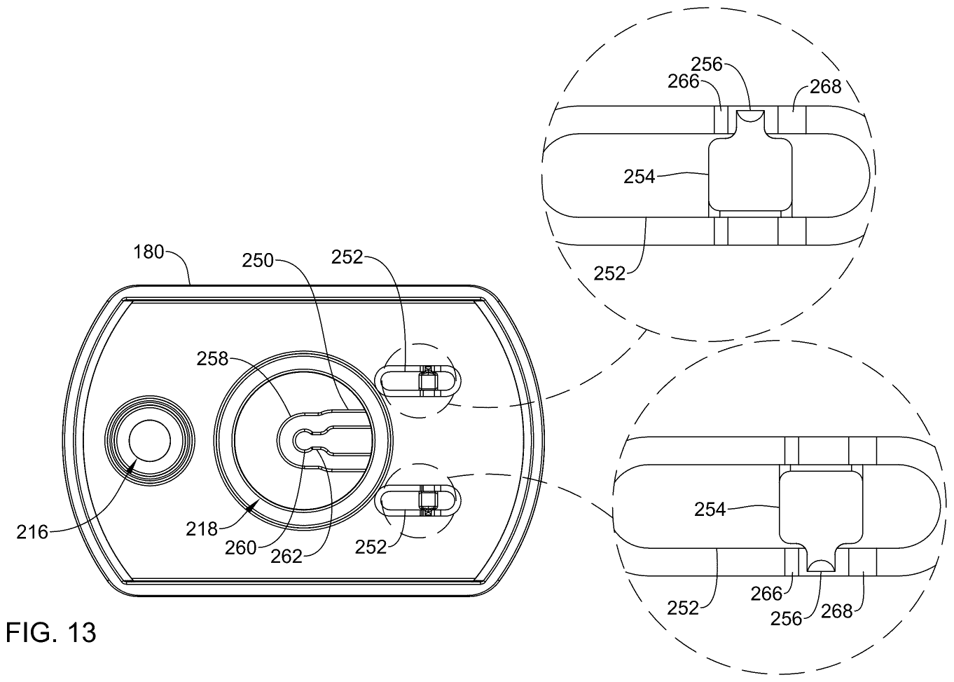

FIG. 13 depicts a schematic second end view (e.g., looking in a proximal direction) of the stabilizer 250 engaged with the cap 180 in a locked position. As discussed, when the stabilizer 250 is in the locked position within the slots 252, the securing portion 260 of the stabilizer 250 may be aligned with (e.g., co-axial with) a central axis extending through the first port entry opening 216, but other suitable positioning of the securing portion 260 when the stabilizer 250 is in a locked position is contemplated.

The cap 180 may include one or features that are configured to facilitate positioning the stabilizer 250 in the locked position and maintaining the stabilizer 250 in the locked position. In some examples, the cap 180 may include one or more bumps or protrusions proximate the slot 252 and configured to facilitate engaging the stabilizer 250 as the stabilizer 250 transitions between the unlocked position and the locked position. In some cases, the one or more bumps or protrusion may prevent or mitigate unintentional adjustment from the locked position to the unlocked position. For example, once the legs 254 and/or the feet 256 of the stabilizer 250 are inserted into the slots 252, the stabilizer 250 may be adjusted between the unlocked position and the locked position by moving the stabilizer 250 to the side such that the foot 256 inserted through the slot 252 may engage and pass over the bump or protrusion 266 proximate the slot 252 in which the leg 254 and/or the foot 256 is inserted. Once the feet 256 have passed over the protrusions 266, the stabilizer 250 may be in the locked position and prevented by the bump or protrusion 266 from unintentionally adjusting to the unlocked position.

To facilitate maintaining the securing portion 260 of the stabilizer 250 aligned with the axis of the first port entry opening 216, the cap 180 may include a stop feature configured to limit movement of the securing portion 260 of the stabilizer 250 from moving away from alignment with the central axis or other suitable portion of the first port entry opening 216. In some examples, the protrusion 266 and the stop 268 may be positioned relative to one another to allow some lateral movement of the stabilizer 250 while the stabilizer 250 is in the locked position. In other examples, the protrusion 266 and the stop 268 may be positioned relative to one another such that no lateral movement of the stabilizer 250 is possible when the stabilizer 250 is in the locked position.

Although the protrusion 266 and the stop 268 are depicted and described as maintaining the stabilizer in a desired position (e.g., the locked position), other suitable configurations are contemplated. For example, the cap 180 may include one or more recesses configured to receive the foot 256 of the stabilizer 250 and maintain the stabilizer 250 in the locked position until a user actively adjusts the stabilizer 250 from the locked position to the unlocked position to disengage the stabilizer from the medical device extending through the first port 122.

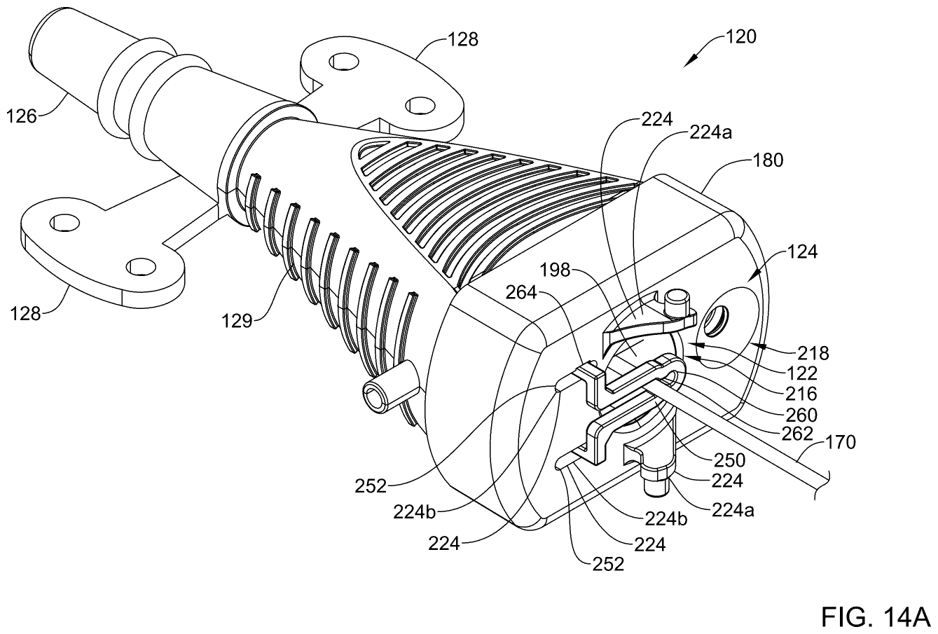

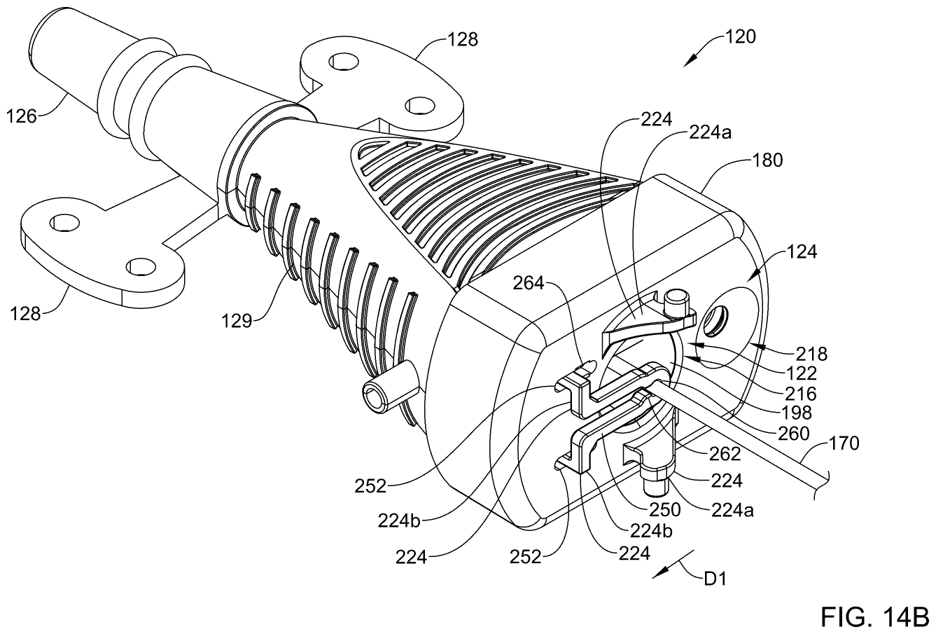

FIGS. 14A and 14B schematically depict adjusting the stabilizer 250 between the unlocked position with a medical device 170 (e.g., an elongate shaft of the medical device) extending therethrough, inserted through the first port 122 (e.g., through the first seal 198 of the first port 122), and advanced through the elongate shaft 114 to a target location, and the locked position with the medical device 170 axially secured at the target location and relative to the stabilizer 250 and/or the hub 120. As depicted in FIG. 14A, the stabilizer 250 is coupled with the hub 120 and in the unlocked position with the legs 254 inserted through the slots 252 and the medical device 170 extending through the arm 258. When the stabilizer 250 is in the unlocked position, the medical device 170 may be located within the arm(s) 258 and to the side of the securing portion 260 and the locking portion 262, but other suitable configurations are contemplated. Although not depicted in FIGS. 14A and 14B, a second medical device may be inserted through the second port 124 and advanced through the elongate shaft 114, as depicted for example in FIG. 7.

To adjust the stabilizer 250 to the locked position, the stabilizer 250 and legs 254 within the slots 252 may be moved laterally in the direction D1. As the stabilizer 250 is moved laterally, the locking portion 262 of the arm(s) 258 may engage or couple the medical device 170 and pass over the medical device 170 until the securing portion 260 of the arm(s) 258 receives the medical device 170. Alternatively or additionally, the stabilizer 250 may be adjusted to the locked position and then the medical device 170 may be moved to the securing portion 260 of the arm(s) 258 such that the medical device 170 is axially secured in a desired position. To adjust the stabilizer 250 from the locked position to the unlocked position and release the medical device 170 from the stabilizer 250, the steps for adjusting the stabilizer 250 from the unlocked position to the locked position may be reversed, while maintaining the medical device 170 in a desired position. Other suitable configurations for adjusting the stabilizer 250 and/or the medical device 170 between the unlocked position and the locked position are contemplated.

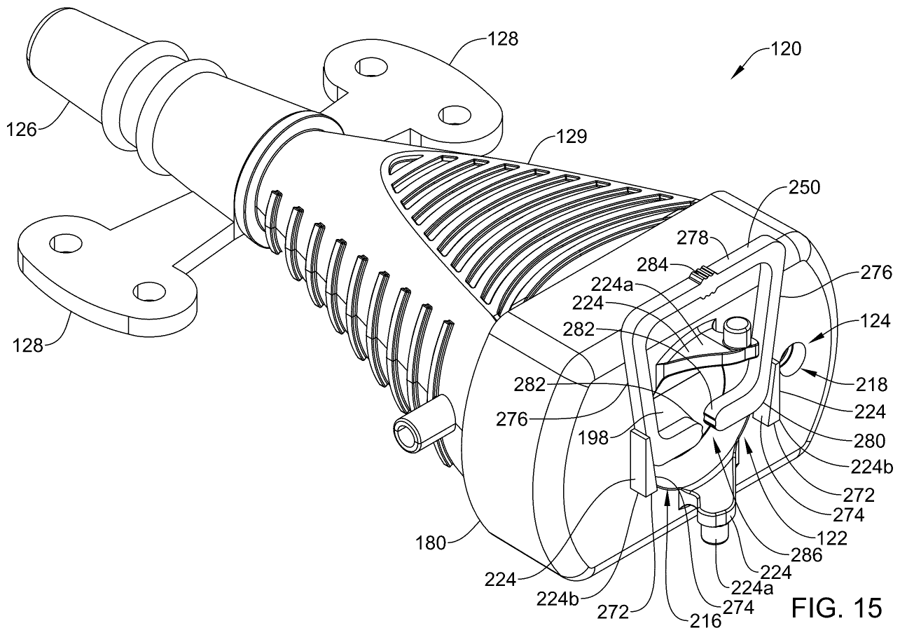

FIG. 15 is a schematic perspective view of an illustrative configuration of the hub 120, with an illustrative configuration of the stabilizer 250 (e.g., a clip, etc.) As depicted in FIG. 15, the hub 120 may include the first port 122 and the second port 124, along with two sets of connectors 224 at or in the proximal facing surface of the hub 120. In some examples, the cap 180 of the hub 120 may include the first set 224a of connectors 224 with the bayonet connector configuration and a configuration of the second set 224b of connectors 224 with an angled ridge connector configuration. In one example, when the first set 224a of connectors 224 and the second set 224b of connectors 224 are utilized as depicted in FIG. 15, the first set 224a of connectors 224 may be configured to couple with a dilator hub (not shown) and the second set 224b of connectors 224 may be configured to couple with the stabilizer 250 (e.g., a clip, etc.)

The second set 224b of the connectors 224 may have any suitable ridge connector configuration that facilitates coupling the stabilizer 250 (e.g., a clip, etc.) with the hub 120 to engage a medical device extending through the first port 122 and/or the second port 124. In some examples, the connectors 224 may include one or more ridges 272 in the cap 180 that are configured to engage and/or receive the stabilizer 250 and couple the stabilizer 250 with the cap 180. In some examples, the ridge(s) 272 may extend proximally from a proximal facing surface of the cap 180. In one example, the second set 224b of the connectors 224 may include two ridges 272 that are spaced from one another on opposing sides of the first port entry opening 216, but other suitable configurations are contemplated.

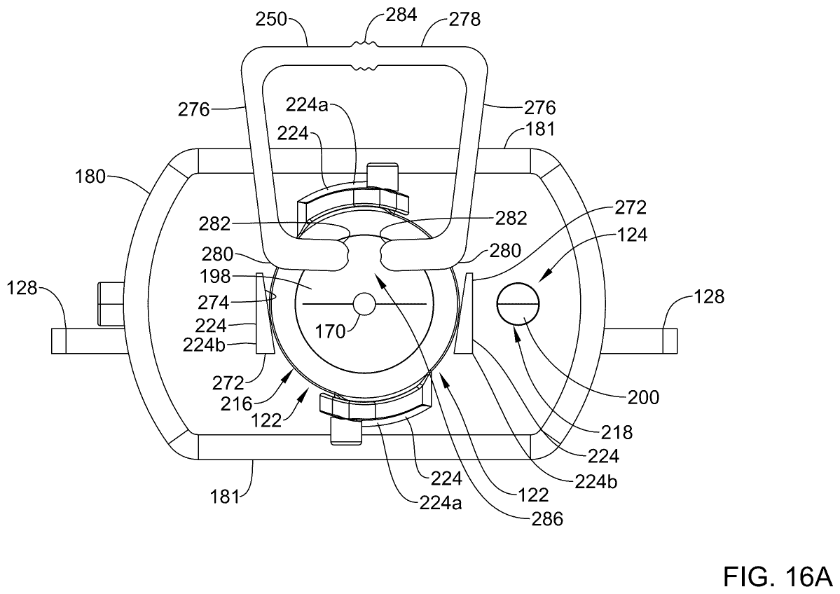

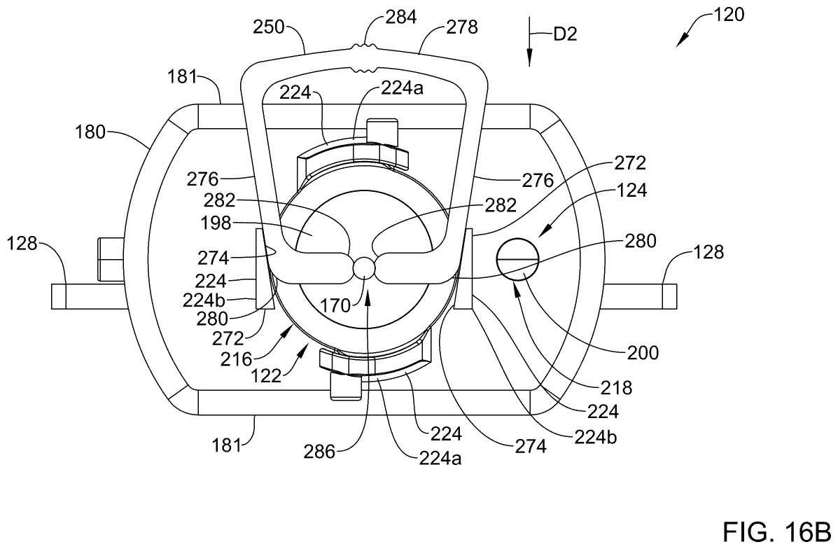

The ridges 272 may have any suitable configuration. In some examples, one or more of the ridges 272 may have a ramped surface 274, where the ramped surface 277 may face the first port entry opening 216, as depicted in FIG. 15. When two ridges 272 are utilized on opposing sides of the first port entry opening 216 and both of the ridges 272 include the ramped surface 274, the ramped surfaces 274 may both angle in a same or similar manner from a first vertical position to a second vertical position, as depicted for example in FIGS. 15-16B. In such an example, a space between the opposing ramped surfaces 274 of the ridges 272 may decrease in a direction from the first vertical position to the second vertical position. Alternatively, when two ridges 272 are utilized, the ramped surface 274 may be omitted on one or both of the ridges 272. In one example, one of the ridges 272 may include the ramped surface 274 and the other ridge 272 may include a straight surface that opposes the ramped surface, but other suitable configurations are contemplated.

The stabilizer 250 configured to engage the ridges 272 may have any suitable configuration configured to couple with the hub 120 (e.g., the cap 180 and/or other suitable component of the hub 120) and engage a medical device extending through one of the ports of the hub 120 (e.g., the first port 122, the second port 124, the third port 132, when included, etc.) In some examples, the stabilizer 250 may be configured to couple with the ridges 272 to engage a medical device extending therethrough when coupled with the second set 224b of connectors. Although the stabilizer 250 may have other suitable configurations, the stabilizer 250 may have or may be a single component clip configuration.

The stabilizer 250 may include one or more arms 276 and a spacer 278 extending between the arms 276, as depicted for example in FIG. 15. In some examples, the one or more arms 276 and the spacer 278 may be entirely or at least partially co-planar. In some examples, the stabilizer 250 may include two arms 276 with the spacer 278 extending between proximal ends of the arms 276. Although other suitable configurations are contemplated, the arms 276 may extend distally from the spacer 278, form an elbow 280 at which the arms 276 are bent toward each other and terminate at distal ends 282, such that an adjustable opening 286 may be located between the distal ends 282 of the arms 276. The adjustable opening 286 may be configured to narrow as outer surfaces of the arms 276 engage the ramped surfaces 274 of the ridges 272 and the distal ends 282 move toward one another. In some examples, the distal ends 282 may have a contoured portion (e.g., rounded portion and/or other suitable contoured portion), as depicted in FIGS. 15-16B, configured to receive or engage a medical device.

The spacer 278 of the stabilizer 250 may include one or more flex components 284. The flex component 284 may be any suitable component or feature of the stabilizer 250 which is designed to bend or flex in response to the arms 276 engaging the ridges 272 (e.g., one or more ramped surfaces 274 of the ridges 272). Alternatively or additionally, the spacer 278 and/or one or more of the arm(s) 276 may be formed from a flexible material that may be configured to bend in response to the arms 276 engaging the ridges 272 and the flex component 284 may be omitted.

The stabilizer 250 may couple with the hub 120 via a friction fit with ridges 272. Alternatively or additionally, stabilizer may couple with the hub 120 via lips extending from the ridges 272, such that as the arms 276 engage the ridges 272, the arms 276 may be located between the proximal facing surface from which the ridges 272 may extend and a distal facing surface of the lips. In some examples, the lip(s) may facilitate mitigating slip-outs of the arms 276 and the stabilizer 250 relative to the ridges 272.

FIGS. 16A and 16B schematically depict adjusting the stabilizer 250 between the unlocked position with the medical device 170 (e.g., an elongate shaft of the medical device) extending therethrough, through the first port 122, and advance through the elongate shaft 114 to a target location and the locked position with the medical device 170 axially secured relative to the stabilizer 250 and/or the hub 120 and the target location. Although not depicted in FIGS. 16A and 16B, a second medical device may be inserted through the second port 124 and advanced through the elongate shaft 114, as depicted for example in FIG. 7.

As depicted in FIG. 16A, the stabilizer 250 is in the unlocked position with the arms 276 spaced from the ramped surfaces 274 and the medical device 170 extending through the first port 122. When the stabilizer 250 is in the unlocked position, the medical device 170 may be axially and/or rotationally adjusted as desired. Although the arms 276 of the stabilizer 250 are disengaged from the ridges in the unlocked position depicted in FIG. 16A, it is contemplated that the arms 276 may engage ridges 272 while still being in the unlocked position, which may facilitate maintaining the stabilizer 250 at the hub 120.

To adjust the stabilizer 250 to the locked position, the stabilizer 250 and the arms 276 may be advanced toward the ridges 272 (e.g., toward the ramped surfaces 274) in the direction D2 and outer surfaces of the arms 276 may engage the ramped surface(s) 274 of the ridges 272, as depicted in FIG. 16B. As the stabilizer 250 is moved in the direction D2, the opening 286 between the distal ends 282 of the arms 276 may narrow and the medical device 170 may be received within the opening 286. The stabilizer 250 may be advanced in the direction D2 until the medical device 170 is received and secured within the opening 286 at which the stabilizer 250 is in the locked position, as depicted in FIG. 16B. In some examples, the arms 276 of the stabilizer 250 and the ramped surface(s) 274 of the ridges 272 may be configured such that the stabilizer 250 is in the locked position and engaging the medical device 170 while the medical device 170 is co-axial with an axis of the first port 122, as depicted for example in FIG. 16B. To adjust the stabilizer 250 from the locked position to the unlocked position and release the medical device 170 from the stabilizer 250, the steps for adjusting the stabilizer 250 from the unlocked position to the locked position may be reversed, while maintaining the medical device 170 in a desired position. Other suitable configurations for adjusting the stabilizer 250 and/or the medical device 170 between the unlocked position and the locked position are contemplated.

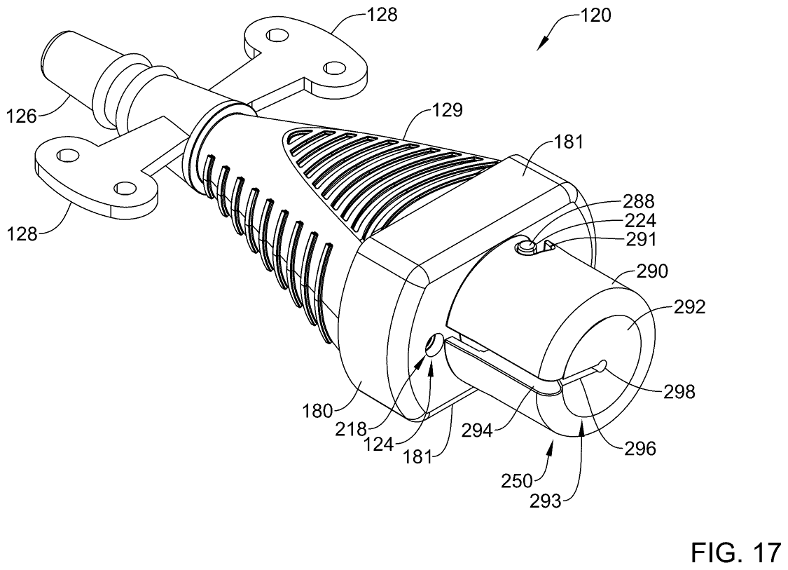

FIG. 17 is a schematic perspective view of an illustrative configuration of the hub 120, with an illustrative configuration of the stabilizer 250. The hub 120 may include the first port 122 (not shown in FIG. 17) and the second port 124, along with a set of connectors 224 (e.g., with only a single connector 224 of the set of connectors 224 depicted in FIG. 17) at or in the proximal facing surface of the hub 120. In some examples, configuration of the connectors 224 have the bayonet connector configuration. In one example, the configuration of the connectors 224 depicted in FIG. 17 may be utilized to couple a dilator hub (not shown) with the hub 120 and/or to couple the stabilizer 250 (e.g., a clip, etc.) with the hub 120.

The connectors 224 in the configuration of the hub 120 depicted in FIG. 17 may have any suitable bayonet connector configuration that facilitates coupling the stabilizer 250 (e.g., a clip, etc.) with the hub 120 to engage a medical device extending through the first port 122 and/or the second port 124. In some examples, the connector 224 may extend proximally from a proximal facing surface of the cap 180 and radially or laterally outward. In some examples, the illustrative configuration of the bayonet connectors 224 may include one or more radial or lateral extensions 288 configured to engage and/or be received in a slot 291 (e.g., and L-shaped slot or other suitable slot) of the stabilizer 250 to couple the stabilizer 250 with the cap 180.

The stabilizer 250 configured to engage the set of connectors 224 may have any suitable configuration configured to couple with the hub 120 (e.g., the cap 180 and/or other suitable component of the hub 120) and engage a medical device extending through one of the ports of the hub 120 (e.g., the first port 122, the second port 124, the third port 132, when included, etc.) In some examples, the stabilizer 250 may be configured to couple with the radial or lateral extensions 288. Although the stabilizer 250 may have other suitable configurations, the stabilizer 250 may have at least two components, which may include a housing 290 (e.g., a stabilizer housing and/or other suitable housing) and a seal 292 (e.g., a stabilizer seal and/or other suitable seal).

The housing 290 may have any suitable configuration for engaging the hub 120, receiving the seal 292, and receiving the medical device. In some examples, the housing 290 may include the slot 291 configured to receive the radial or lateral extension 288 of the connector 224, an opening 293 configured to receive the medical device and the seal 292, and an elongate slot 294 configured to receive the medical device into the opening 293. Other suitable configurations of the housing are contemplated.

The housing 290 may be formed from any suitable material. For example, the housing 290 may be formed from a metallic material, a polymer material, a rigid material, a flexible material, and/or other suitable material. In one example, the housing 290 may be formed from a material that is the same as or similar to a material used to form the cap 180, but other suitable configurations are contemplated.

The seal 292 may have any suitable configuration for engaging the housing 290 and receiving the medical device. In some examples, the seal 292 may include a securing portion or opening 298 (e.g., an axially elongated securing portion or opening) configured to align with the first port entry opening 216 and to engage the medical device and an elongate slot 296 (e.g., a stabilizer seal slot and/or other suitable slot) extending from the securing portion or opening 298 to an outer circumference of the seal 292 and extending along a length of the seal 292. The slot 296 may be configured to receive the medical device into the opening 298. In one example, the elongate slot 296 of the seal 292 may be configured to align with the slot 294 of the housing 290 to facilitate receiving the medical device. Once the medical device has been received in the securing portion or opening 298, the seal 292 may be adjusted to misalign the elongate slot 296 in the seal 292 relative to the elongate slot 294 in the housing 290. Other suitable configurations are contemplated.

The securing portion or opening 298 of the seal 292 may have any suitable diameter. In some examples, the diameter of the securing portion or opening 298 may be configured to have a diameter that is equal to or less than a diameter of the medical device received therein to facilitate applying a compressive force to the medical device for axially and/or rotationally fixing (e.g., axially and/or rotationally fixing) the medical device relative to the cap 180 and/or the hub 120. Alternatively or additionally, at least a portion of the securing portion or opening 298 may have a diameter that is greater than a diameter of the medical device received in the seal 292.

The seal 292 of the stabilizer 250 may be formed from any suitable material. In some examples, the seal 292 may be formed from material including, but not limited to, elastomeric material, resilient material, compressible material, flexible material, silicone material, polyurethane material, and or other suitable materials. In one example, the seal 292 may be or may be made from a compressible material to form a compressible seal around the medical device when inserted in the housing 290, but other suitable configurations are contemplated.

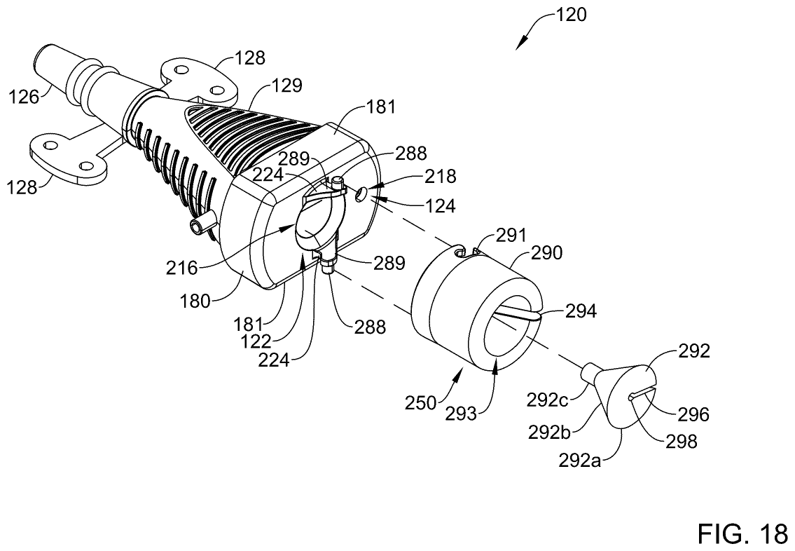

FIG. 18 is a schematic perspective view of an illustrative configuration of the hub 120 depicted in FIG. 17, with an illustrative configuration of a stabilizer 250 exploded out from the hub 120. As depicted in FIG. 18, the hub 120 may include the first port 122 and the second port 124, along with the set of connectors 224 having the bayonet configuration. In some examples, the set of connectors 224 may have at least two connectors 224 that are on opposite sides of the first port entry opening 216 and configured to receive and engage the stabilizer 250 at the first port 122. Each connector 224 of or in a proximal facing surface of the hub 120 may include a proximal extension 289 extending proximally from the proximal facing surface of the cap 180 and/or from one or more other suitable locations and the radial or lateral extension 288 may extend radially or laterally outward (e.g., as depicted in FIG. 18) or inward from the proximal extension 289. In one example, the radial or lateral extensions 288 of the connectors 224 may be coaxial with one another to facilitate a bayonet connection with associated slots 291 in the housing 290. For example, two of the slots 291 of the housing 290 may extend proximally into the housing 290 from a distal end of the housing 290 and may be or may be approximately one hundred eighty degrees apart and configured to receive the coaxial radial or lateral extensions 288 through the distal end of the housing 290 and then couple with cap 180 by rotating the stabilizer 250 or the hub 120 relative to the other to engage the stabilizer 250 with the hub 120.

The opening 293 of the housing 290 may have any suitable configuration. Although other configurations are contemplated, the opening 293 may be axially elongated and may extend from a proximal circular opening and taper distally to a constant diameter cylindrical portion. Other suitable configurations of the opening 293 of the housing 290 are contemplated.

The seal 292 may have any suitable configuration. In some examples, the outer surface of the seal 292 may have a similar shape as the opening 293 of the housing 290 to facilitate being received in and engaging the opening 293. For example, the seal 292 may have an elongated axial configuration extending distally from a proximal first portion 292a having circular shape, to second portion 292b that tapers distally to a third portion 292c having a cylindrical shape. The securing portion or opening 298 of the seal 292 may be at a center of the seal 292 and extend a length of the seal 292. Other suitable seal configurations are contemplated.

In some examples, the seal 292 may include one or more projections and/or or recesses configured to engage recesses and/or projections, respectively, on an inner surface of the housing 290 (e.g., along the opening 293). In one example, the distal, cylindrical portion of the seal 292 may include one or more recesses (e.g., a circumferential or an annular recess and/or other suitable recess) configured to receive and/or engage one or more projections (e.g., a circumferential or annular projection and/or other suitable projection) on the inner surface of the housing 290.

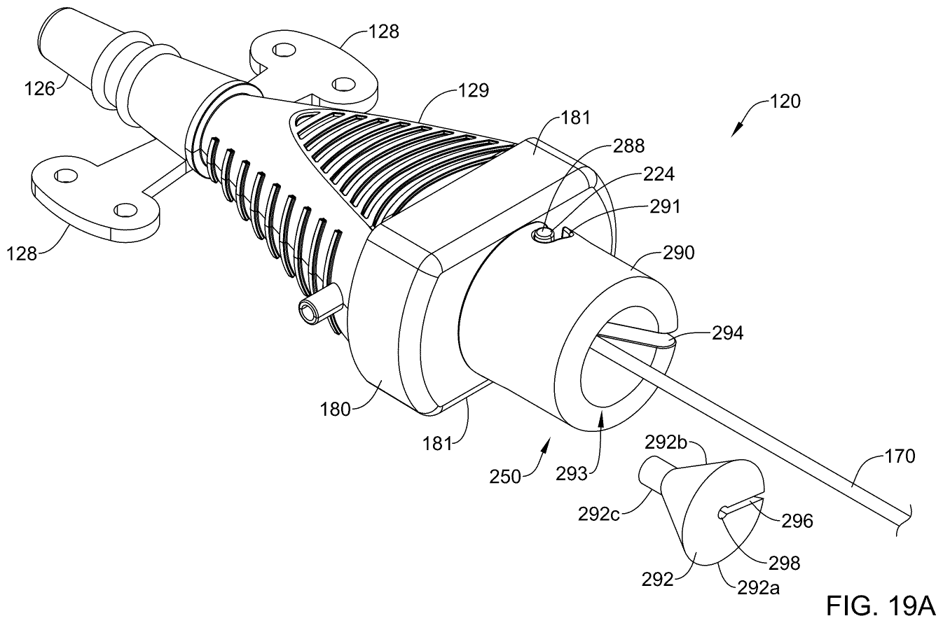

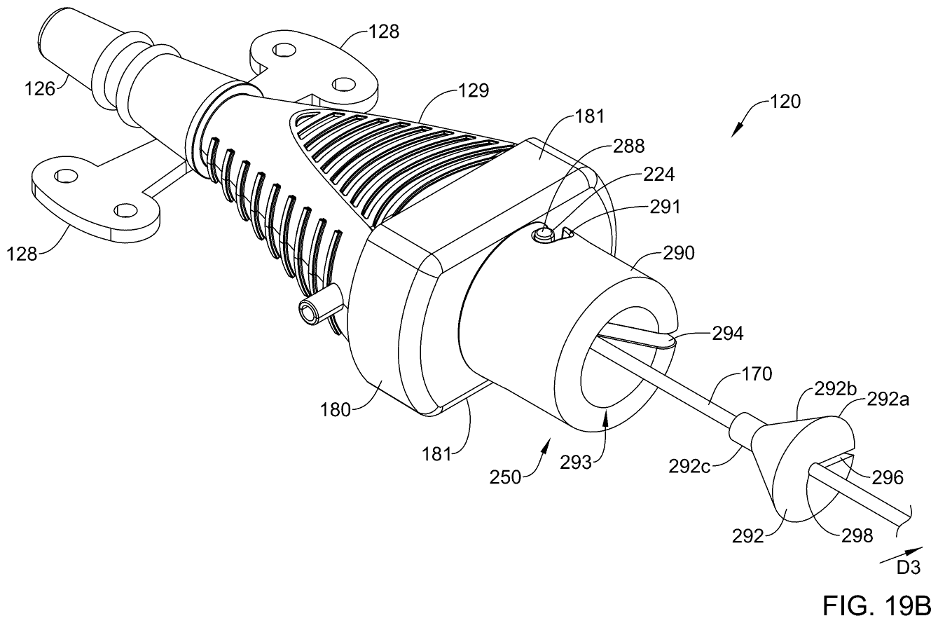

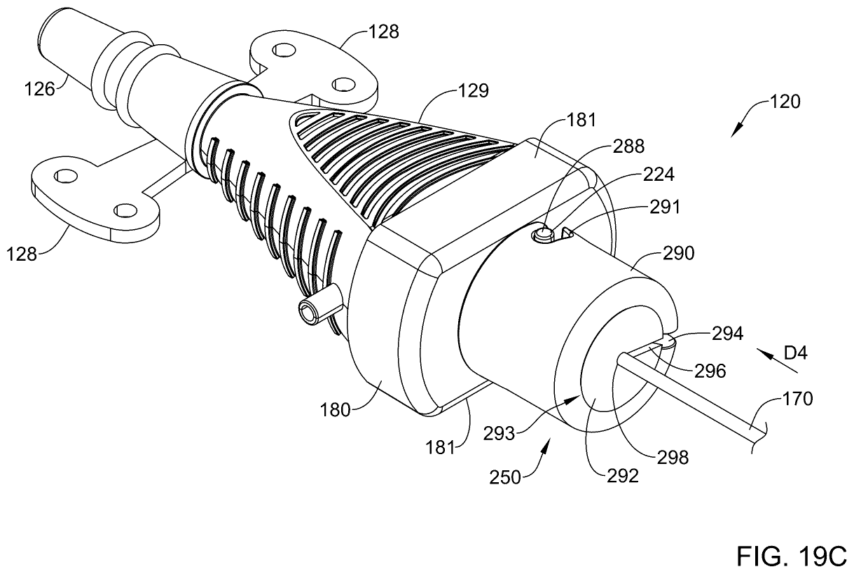

FIGS. 19A-19C schematically depict adjusting the stabilizer 250 between the unlocked position with a medical device 170 (e.g., an elongate shaft of the medical device) extending therethrough and through the first port 122 (not shown in FIGS. 19A-19C) and the locked position with the medical device 170 axially secured relative to the stabilizer 250 and/or the hub 120. Although not depicted in FIGS. 19A-19C, a second medical device may be inserted through the second port 124 and advanced through the elongate shaft 114, as depicted in FIG. 7.

As depicted in FIG. 19A, the stabilizer 250 is in the unlocked position, which the housing 290 of the stabilizer 250 coupled with the cap 180 via the connectors 224, the medical device 170 extending through the housing 290, and the seal 292 separated from the housing 290. The medical device 170 may be received in the housing 290 axially or radially through the slot 294 prior to and/or while the housing 290 is coupled with the cap 180, for example. When the stabilizer 250 is in the unlocked position, the medical device 170 may be axial, rotationally, and/or laterally adjusted within and/or about the opening 293 of the housing 290, but other suitable configurations are contemplated.

To initiate adjusting the stabilizer 250 to the locked position, the medical device 170 may be received in the securing portion or opening 298 of the seal 292, for example as depicted in FIG. 19B. In some examples, the seal 292 may be flexible and/or resilient such that the medical device 170 may be received in the securing portion or opening 298 by sliding the seal 292 in the direction D3 over the medical device 170 such that the medical device 170 is slid through the elongate slot 296 to the securing portion or opening 298. In some examples, the medical device may extend through the seal 292 from the first portion 292a having the circular cross-section shape, through the second portion 292b having an axial distal taper, and the third portion 292c having a cylindrical shape.

To adjust the stabilizer 250 to the locked position and axially and rotationally secure the medical device 170 relative to the stabilizer 250 and/or the hub 120, the seal 292 may be advanced distally in the direction of D4 along the medical device 170 until the seal 292 is fully inserted in the opening 293 of the housing 290, as depicted for example in FIG. 19C. Alternatively or additionally, the seal 292 may be inserted into the housing 290 and/or coupled with the medical device 170 in one or more other suitable steps or manners, as desired. When the seal 292 is fully inserted in the opening 293, a proximal end of the seal 292 may be inset from, extend proximally from, align with, or be co-planar with a proximal end of the housing 290.

Once the seal 292 is inserted into the housing 290, the housing 290 may apply a compressive force on the seal 292, which may maintain the seal 292 in the housing 290. Further, the compressive force acting on the seal 292 from the coupling between the housing 290 and the seal 292 may apply a sufficient compressive force on the medical device 170 to prevent or mitigate unintentional rotational and/or axial movement of the medical device 170 relative to the stabilizer 250 and/or the hub 120 without damaging the medical device 170.

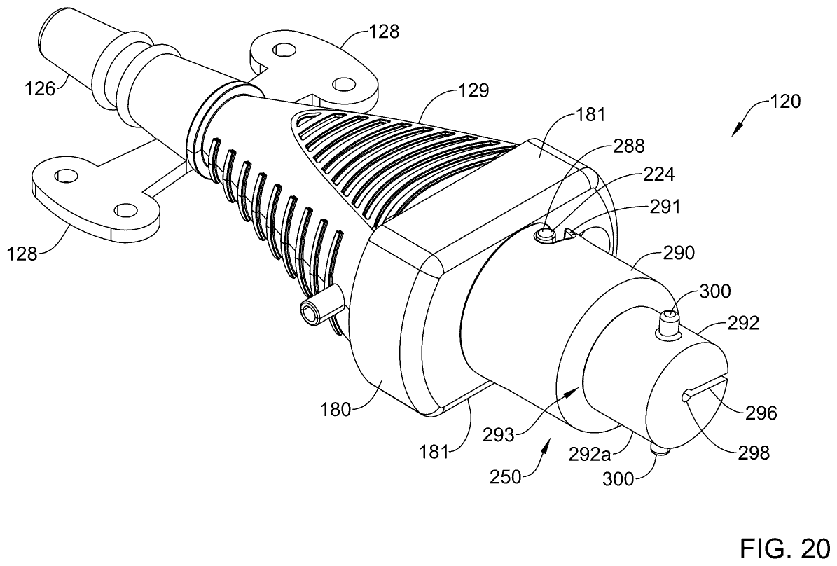

FIG. 20 is a schematic perspective view of the illustrative configuration of the hub 120 depicted in FIG. 17, with an illustrative configuration of the stabilizer 250. The illustrative configuration of the stabilizer 250 depicted in FIG. 20 may have some similar to features of the configuration depicted in and described with respect to FIGS. 17-19C, such similar features that have been described in detail herein are not further described here. For example, the configuration of the stabilizer 250 depicted in FIG. 20 may include one or more slots 291 at a distal end of the housing 290 that are configured to couple with radial or lateral extensions 288 of the connectors 224 via a bayonet connection or coupling. Further, the housing 290 may include the opening 293 and the slot 294. The housing 290 may receive and engage the seal 292 at the opening 293.

When the seal 292 has been fully received in the opening 293, as depicted in FIG. 20, the seal 292 may extend proximally from a proximal end of the housing 290. In one example, the first portion 292a of the seal 292 may be cylindrical or have one or more other suitable shapes or configurations extending proximally from the proximal end of the housing 290, as depicted for example in FIG. 20.

The seal 292 may include one or more seal projections 300 extending radially outward or laterally outward from an outer surface of the seal 292. The seal projections 300 may co-axial with one another, but other suitable configurations are contemplated. When included, the seal projections 300 may be utilized to apply forces to the seal 292 in a distal direction to facilitate inserting the seal 292 into the opening 293 of the housing 290 and adjusting the stabilizer 250 from the unlocked position to the locked position, to engage the housing 290 with the connectors 224, to rotate the seal 292 within the housing 290, to apply forces to the seal 292 in a proximal direction to facilitate removing the seal 292 from the housing 290 and adjusting the stabilizer 250 from the locked position to the unlocked position, and/or to apply other forces to the seal 292.

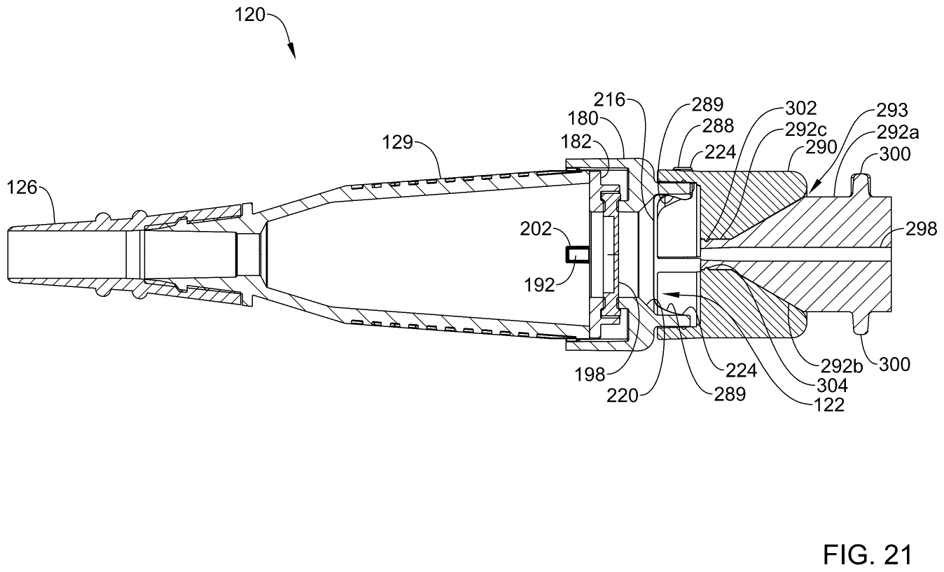

FIG. 21 depicts a schematic cross-section view of the hub 120 coupled with the configuration of the stabilizer 250 depicted in FIG. 20. As depicted in FIG. 21, the stabilizer 250 may include a securing or locking system for coupling the seal 292 with the housing 290 when the stabilizer 250 is in the locked position. In some examples, the securing or locking system may include one or more mating components or features on the interior surface of the housing 290 and/or the exterior surface of the seal 292 to releasably engage and/or fix the seal 292 with and/or relative to the housing 290. For example, the securing or locking system may include, among additional or alternative components, a projection 302 on an interior surface of the housing 290 that is configured to engage a recess 304 on an exterior surface of the seal 292 when the seal 292 is fully inserted into the housing 290 to create an interference fit. In one example, the projection 302 may be a circumferential projection on the interior surface of the housing and the recess 304 may be a circumferential recess on the exterior surface of the third portion 292c of the seal 292, but other suitable configurations are contemplated.

This disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An introducer sheath, comprising:
a valve hub having a proximal end defining a first port having a first port entry opening and a second port having a second port entry opening;
an elongate shaft extending from the valve hub;
a first seal, the first seal is disposed at the first port and is aligned with the first port entry opening; and
a second seal, the second seal is disposed at the second port and is aligned with the second port entry opening, and
wherein the first port is parallel to the second port.

2. The introducer sheath of claim 1, wherein the first seal is co-planar with the second seal.

3. The introducer sheath of claim 1, wherein the first port entry opening is co-planar with the second port entry opening.

4. The introducer sheath of claim 1, further comprising:
a third port defined by the valve hub, the third port having a third port entry opening;
a third seal, the third seal is disposed at the third port and is aligned with the third port entry opening, and
wherein the third port is parallel with the first port.

5. The introducer sheath of claim 1, wherein the first port entry opening has a first diameter and the second port entry opening has a second diameter that is smaller than the first diameter.

6. The introducer sheath of claim 1, wherein the first port entry opening is co-axial with the elongate shaft and the second port entry opening is laterally offset from the first port entry opening.

7. The introducer sheath of claim 1, wherein one or both of the first port entry opening and the second port entry opening include a chamfer narrowing in a distal direction toward the elongate shaft.

8. The introducer sheath of claim 1, wherein the valve hub comprises:
a body coupled with the elongate shaft; and
a cap coupled with the body, and
wherein the cap defines the first port entry opening and the second port entry opening.

9. The introducer sheath of claim 8, further comprising:
a seal guide positioned between the body and the cap, and
wherein the seal guide is configured to receive the first seal and the second seal to align the first seal with the first port entry opening and align the second seal with the second port entry opening.

10. The introducer sheath of claim 8, further comprising:
one or more walls within the body, and
wherein the one or more walls are configured to direct one or more medical devices received in one or both of the first port and the second port toward a lumen of the elongate shaft.

11. The introducer sheath of claim 1, further comprising:
one or more connectors defined by the valve hub, and
wherein the one or more connectors are configured to engage a stabilizer configured to receive and axially fix a medical device extending through one or both of the first port and the second port.

12. An introducer sheath assembly, comprising:
a valve hub having a proximal end defining a first port having a first port entry opening and a second port having a second port entry opening;
an elongate shaft extending from the valve hub; and
a stabilizer configured to couple with the valve hub and engage a medical device extending through the first port entry opening, and
wherein the first port is parallel to the second port.

13. The introducer sheath assembly of claim 12, further comprising:
one or more slots in a proximal facing surface of the valve hub, and
wherein the stabilizer comprises legs and a stabilizer opening, and the legs are configured to be secured in the one or more slots and the stabilizer opening is configured to align with the first port entry opening when the legs are secured in the one or more slots.

14. The introducer sheath assembly of claim 12, further comprising:
two or more ridges on opposing sides of the first port entry opening, each of the ridges including a ramped surface facing the first port entry opening, and
wherein the stabilizer comprises a first arm having a first distal end and a second arm having a second distal end that defines an adjustable opening with the first distal end, and the adjustable opening is configured to narrow as outer surfaces of the first arm and the second arm engage the two or more ridges.

15. The introducer sheath assembly of claim 12, further comprising:
one or more extensions proximate the first port entry opening, and
wherein the stabilizer comprises a stabilizer housing configured to couple with the one or more extensions.

16. The introducer sheath assembly of claim 15, wherein the stabilizer comprises a stabilizer seal having a stabilizer seal opening configured to align with the first port entry opening and a stabilizer seal slot extending from the stabilizer seal opening to an outer circumference of the stabilizer, and the stabilizer is configured to axially fix the medical device when the stabilizer seal is coupled with the stabilizer housing and the medical device is extending through the stabilizer seal opening.

17. A method of positioning one or more medical devices through an introducer sheath having a valve hub, the method comprising:
inserting a first medical device through a first seal of a first port of the valve hub;
advancing the first medical device through an elongate shaft of the introducer sheath;
inserting a second medical device through a second seal of a second port of the valve hub, the second port is parallel with the first port; and
advancing the second medical device through the elongate shaft of the introducer sheath.

18. The method of claim 17, further comprising:
axially fixing the first medical device relative valve hub by coupling a stabilizer with the first medical device inserted through the first seal and advanced through the introducer sheath.

19. The method of claim 17, further comprising:
coupling a stabilizer with the valve hub.

20. The method of claim 19, further comprising:
adjusting the stabilizer coupled with the valve hub from an unlocked position to a locked position to axially fix the first medical device relative to the valve hub.
